SEQUENCE LISTING (45 sequences)

SEQ ID NO: 1 - DNA, 1860 bp, Artificial Sequence
  Artificially Synthesized Recombinant cdk5-REV-TagGFP Biosensor
atgcagaaatacgagaaactggaaaagattggggaaggcacctacggaactgtgttcaaggccaaaaaccgggagactcatgagatcgtggctctgaaacgggtgaggctggatgacgatgatgagggtgtgccgagttccgccctccgggagatctgcctactcaaggagctgaagcacaagaacatcgtcaggcttcatgacgtcctgcacagcgacaagaagctgactttggtttttgaattctgtgaccaggacctgaagaagtattttgacagttgcaatggtgacctcgatcctgagattgtaaagtcattcctcttccagctactaaaagggctgggattctgtcatagccgcaatgtgctacacagggacctgaagccccagaacctgctaataaacaggaatggggagctgaaattggctgattttggcctggctcgagcctttgggattcccgtccgctgttactcagctgaggtggtcacactgtggtaccgcccaccggatgtcctctttggggccaagctgtactccacgtccatcgacatgtggtcagccggctgcatctttgcagagctggccaatgctgggcggcctctttttcccggcaatgatgtcgatgaccagttgaagaggatcttccgactgctggggacgcccaccgaggagcagtggccctctatgaccaagctgccagactataagccctatccgatgtacccggccacaacatccctggtgaacgtcgtgcccaaactcaatgccacagggagggatctgctgcagaaccttctgaagtgtaaccctgtccagcgtatctcagcagaagaggccctgcagcacccctacttctccgacttctgtccgcccacgccgtcgacggtacccatggcaggaagaagcggagacagcgacgaagagctcatcagaacagtcagactcatcaagcttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagcccccccgggatccaccggtcgccaccatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatcctcttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatcatgcccgacaaggccaacaacggcctggaggtgaacttcaagacccgccacaacatcgagggcggcggcgtgcagctggccgaccactaccagaccaacgtgcccctgggcgacggccccgtgctgatccccatcaaccactacctgagcactcagaccgccatcagcaaggaccgcaacgaggcccgcgaccacatggtgctcctggagtccttcagcgcctgctgccacacccacggcatggacgagctgtacaggtaa

SEQ ID NO: 2 - protein, 619 aa, Artificial Sequence
  Artificially Synthesized Recombinant cdk5-REV-TagGFP Biosensor
MQKYEKLEKIGEGTYGTVFKAKNRETHEIVALKRVRLDDDDEGVPSSALREICLLKELKHKNIVRLHDVLHSDKKLTLVFEFCDQDLKKYFDSCNGDLDPEIVKSFLFQLLKGLGFCHSRNVLHRDLKPQNLLINRNGELKLADFGLARAFGIPVRCYSAEVVTLWYRPPDVLFGAKLYSTSIDMWSAGCIFAELANAGRPLFPGNDVDDQLKRIFRLLGTPTEEQWPSMTKLPDYKPYPMYPATTSLVNVVPKLNATGRDLLQNLLKCNPVQRISAEEALQHPYFSDFCPPTPSTVPMAGRSGDSDEELIRTVRLIKLLYQSNPPPNPEGTRQARRNRRRRWRERQRQIHSISERILSTYLGRSAEPVPLQPPRDPPVATMSGGEELFAGIVPVLIELDGDVHGHKFSVRGEGEGDADYGKLEIKFICTTGKLPVPWPTLVTTLCYGIQCFARYPEHMKMNDFFKSAMPEGYIQERTILFQDDGKYKTRGEVKFEGDTLVNRIELKGKDFKEDGNILGHKLEYSFNSHNVYIMPDKANNGLEVNFKTRHNIEGGGVQLADHYQTNVPLGDGPVLIPINHYLSTQTAISKDRNEARDHMVLLESFSACCHTHGMDELYR

SEQ ID NO: 3 - protein, 238 aa, Artificial Sequence
  Artificially Synthesized Recombinant TagGFP
MSGGEELFAGIVPVLIELDGDVHGHKFSVRGEGEGDADYGKLEIKFICTTGKLPVPWPTLVTTLCYGIQCFARYPEHMKMNDFFKSAMPEGYIQERTILFQDDGKYKTRGEVKFEGDTLVNRIELKGKDFKEDGNILGHKLEYSFNSHNVYIMPDKANNGLEVNFKTRHNIEGGGVQLADHYQTNVPLGDGPVLIPINHYLSTQTAISKDRNEARDHMVLLESFSACCHTHGMDELYR

SEQ ID NO: 4 - protein, 74 aa, Artificial Sequence
  artificially synthesized recombinant rev
MAGRSGDSDEELIRTVRLIKLLYQSNPPPNPEGTRQARRNRRRRWRERQRQIHSISERILSTYLGRSAEPVPLQ

SEQ ID NO: 5 - protein, 292 aa, Artificial Sequence
  artificially synthesized recombinant cdk5
MQKYEKLEKIGEGTYGTVFKAKNRETHEIVALKRVRLDDDDEGVPSSALREICLLKELKHKNIVRLHDVLHSDKKLTLVFEFCDQDLKKYFDSCNGDLDPEIVKSFLFQLLKGLGFCHSRNVLHRDLKPQNLLINRNGELKLADFGLARAFGIPVRCYSAEVVTLWYRPPDVLFGAKLYSTSIDMWSAGCIFAELANAGRPLFPGNDVDDQLKRIFRLLGTPTEEQWPSMTKLPDYKPYPMYPATTSLVNVVPKLNATGRDLLQNLLKCNPVQRISAEEALQHPYFSDFCPP

SEQ ID NO: 6 - DNA, 1839 bp, Artificial Sequence
  artificially synthesized recombinant TagRFP-NES/NLS-p35 biosensor
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaattccggactcagatctcgagcccctcagactccactgcacaccagccgtgtcctgaaggaggacaaggaacgatgggaggatgtcaaggaggagatgaccagtgccttggccacgatgtgtgttgactatgagcagatcaagataaagaagatagaagacgcatccaaccctctgcttctcaagaggcggaagaaatcgaattccatgggcacggtgctgtccctgtctcccagctaccggaaggccacgctgtttgaggatggcgcggccaccgtgggccactatacggccgtacagaacagcaagaacgccaaggacaagaacctgaagcgccactccatcatctccgtgctgccttggaagagaatcgtggccgtgtcggccaagaagaagaactccaagaaggtgcagcccaacagcagctaccagaacaacatcacgcacctcaacaatgagaacctgaagaagtcgctgtcgtgcgccaacctgtccacattcgcccagcccccaccggcccagccgcctgcacccccggccagccagctctcgggttcccagaccgggggctcctcctcagtcaagaaagcccctcaccctgccgtcacctccgcagggacgcccaaacgggtcatcgtccaggcgtccaccagtgagctgcttcgctgcctgggtgagtttctctgccgccggtgctaccgcctgaagcacctgtcccccacggaccccgtgctctggctgcgcagcgtggaccgctcgctgcttctgcagggctggcaggaccagggcttcatcacgccggccaacgtggtcttcctctacatgctctgcagggatgttatctcctccgaggtgggctcggatcacgagctccaggccgtcctgctgacatgcctgtacctctcctactcctacatgggcaacgagatctcctacccgctcaagcccttcctggtggagagctgcaaggaggccttttgggaccgttgcctctctgtcatcaacctcatgagctcaaagatgctgcagataaatgccgacccacactacttcacacaggtcttctccgacctgaagaacgagagcggccaggaggacaagaagcggctcctcctaggcctggatcggtga

SEQ ID NO: 7 - protein, 612 aa, Artificial Sequence
  artificially synthesized recombinant TagRFP-NES/NLS-p35 biosensor
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRSDMALKLVGGGHLICNFKTTYRSKKPAKNLKMPGVYYVDHRLERIKEADKETYVEQHEVAVARYCDLPSKLGHKLNSGLRSRAPQTPLHTSRVLKEDKERWEDVKEEMTSALATMCVDYEQIKIKKIEDASNPLLLKRRKKSNSMGTVLSLSPSYRKATLFEDGAATVGHYTAVQNSKNAKDKNLKRHSIISVLPWKRIVAVSAKKKNSKKVQPNSSYQNNITHLNNENLKKSLSCANLSTFAQPPPAQPPAPPASQLSGSQTGGSSSVKKAPHPAVTSAGTPKRVIVQASTSELLRCLGEFLCRRCYRLKHLSPTDPVLWLRSVDRSLLLQGWQDQGFITPANVVFLYMLCRDVISSEVGSDHELQAVLLTCLYLSYSYMGNEISYPLKPFLVESCKEAFWDRCLSVINLMSSKMLQINADPHYFTQVFSDLKNESGQEDKKRLLLGLDR

SEQ ID NO: 8 - protein, 237 aa, Artificial Sequence
  artificially synthesized recombinant TagRFP
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRSDMALKLVGGGHLICNFKTTYRSKKPAKNLKMPGVYYVDHRLERIKEADKETYVEQHEVAVARYCDLPSKLGHKLN

SEQ ID NO: 9 - protein, 58 aa, Artificial Sequence
  artificially synthesized recombinant NES/NLS localization signal from MK2
PQTPLHTSRVLKEDKERWEDVKEEMTSALATMCVDYEQIKIKKIEDASNPLLLKRRKK

SEQ ID NO: 10 - protein, 307 aa, Artificial Sequence
  artificially synthesized recombinant p35
MGTVLSLSPSYRKATLFEDGAATVGHYTAVQNSKNAKDKNLKRHSIISVLPWKRIVAVSAKKKNSKKVQPNSSYQNNITHLNNENLKKSLSCANLSTFAQPPPAQPPAPPASQLSGSQTGGSSSVKKAPHPAVTSAGTPKRVIVQASTSELLRCLGEFLCRRCYRLKHLSPTDPVLWLRSVDRSLLLQGWQDQGFITPANVVFLYMLCRDVISSEVGSDHELQAVLLTCLYLSYSYMGNEISYPLKPFLVESCKEAFWDRCLSVINLMSSKMLQINADPHYFTQVFSDLKNESGQEDKKRLLLGLDR

SEQ ID NO: 11 - DNA, 1383 bp, Artificial Sequence
  artificially synthesized recombinant GFP-rev-p53 biosensor
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacctcgagatggcaggaagaagcggagacagcgacgaagagctcatcagaacagtcagactcatcaagcttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctgcagtcgacggtaccgcgggcccgggatccaccggtcgccaccatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatcctcttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatcatgcccgacaaggccaacaacggcctggaggtgaacttcaagacccgccacaacatcgagggcggcggcgtgcagctggccgaccactaccagaccaacgtgcccctgggcgacggccccgtgctgatccccatcaaccactacctgagcactcagaccgccatcagcaaggaccgcaacgaggcccgcgaccacatggtgctcctggagtccttcagcgcctgctgccacacccacggcatggacgagctgtacaggtaa

SEQ ID NO: 12 - protein, 460 aa, Artificial Sequence [sequence omitted]

SEQ ID NO: 13 - protein, 131 aa, Artificial Sequence
  artificially synthesized recombinant p53
MEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALN

SEQ ID NO: 14 - DNA, 1299 bp, Artificial Sequence
  artificially synthesized recombinant JRED-NLS/NES-HDM2 biosensor
atggacgaggatggttcagagggcggccccgccctgttccagagcgacatgaccttcaaaatcttcatcgacggcgaggtgaacggccagaagttcaccatcgtggccgacggcagcagcaagttcccccacggcgacttcaacgtgcacgccgtgtgcgagaccggcaagctgcccatgagctggaagcccatctgccacctgatccagtacggcgagcccttcttcgcccgctaccccaacggcatcagccacttcgcccaggagtgcttccccgagggcctgagcatcgaccgcaccgtgcgcttcgagaacgacggcaccatgaccagccaccacacctacgagctggacggcacctgcgtggtcagccgcatcaccgtgaactgcgacggcttccagcccgacggccccatcatgcgcgaccagctggtggacatcctgcccaacgagacccacatgttcccccacggccccaacgccgtgcgccagctggccttcatcggcttcaccaccgccgacggcggcctgatgatgggccacttcgacagcaagatgaccttcaacggcagccgcgccatcaagatccccggcccccacttcgtgaccatcatcaccaagcagatgagggacaccagcgacaagcgcgaccacgtgtgccagcgcgaggtgacctacgcccacagcgtgccccgcatcaccagcgccatcggtagcgacgaggattccggactcagatctcgagctcaagcttcgaattcccctcagactccactgcacaccagccgtgtcctgaaggaggacaaggaacgatgggaggatgtcaaggaggagatgaccagtgccttggccacgatgtgtgttgactatgagcagatcaagataaagaagatagaagacgcatccaaccctctgcttctcaagaggcggaagaaactcgagatgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcataa

SEQ ID NO: 15 - protein, 432 aa, Artificial Sequence [sequence omitted]

SEQ ID NO: 16 - protein, 242 aa, Artificial Sequence
  artificially synthesized recombinant JRED
MDEDGSEGGPALFQSDMTFKIFIDGEVNGQKFTIVADGSSKFPHGDFNVHAVCETGKLPMSWKPICHLIQYGEPFFARYPNGISHFAQECFPEGLSIDRTVRFENDGTMTSHHTYELDGTCVVSRITVNCDGFQPDGPIMRDQLVDILPNETHMFPHGPNAVRQLAFIGFTTADGGLMMGHFDSKMTFNGSRAIKIPGPHFVTIITKQMRDTSDKRDHVCQREVTYAHSVPRITSAIGSDED

SEQ ID NO: 17 - protein, 118 aa, Artificial Sequence
  artificially synthesized recombinant HDM2 (aa 1-118)
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDS

SEQ ID NO: 18 - DNA, 1284 bp, Artificial Sequence
  artificially synthesized recombinant TagRFP-NES/NLS-HDM2 biosensor
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaattccggactcagatctcgagctcaagcttcgaattcccctcagactccactgcacaccagccgtgtcctgaaggaggacaaggaacgatgggaggatgtcaaggaggagatgaccagtgccttggccacgatgtgtgttgactatgagcagatcaagataaagaagatagaagacgcatccaaccctctgcttctcaagaggcggaagaaactcgagatgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcataa

SEQ ID NO: 19 - protein, 427 aa, Artificial Sequence
  artificially synthesized recombinant TagRFP-NES/NLS-HDM2 biosensor
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRSDMALKLVGGGHLICNFKTTYRSKKPAKNLKMPGVYYVDHRLERIKEADKETYVEQHEVAVARYCDLPSKLGHKLNSGLRSRAQASNSPQTPLHTSRVLKEDKERWEDVKEEMTSALATMCVDYEQIKIKKIEDASNPLLLKRRKKLEMCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDS

SEQ ID NO: 20 - DNA, 621 bp, Artificial Sequence
  artificially synthesized recombinant p53 (1-131)-REV (1-74) biosensor
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacctcgagatggcaggaagaagcggagacagcgacgaagagctcatcagaacagtcagactcatcaagcttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcag

SEQ ID NO: 21 - protein, 207 aa, Artificial Sequence
  artificially synthesized recombinant p53 (1-131)-REV (1-74) biosensor
MEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNLEMAGRSGDSDEELIRTVRLIKLLYQSNPPPNPEGTRQARRNRRRRWRERQRQIHSISERILSTYLGRSAEPVPLQ

SEQ ID NO: 22 - DNA, 537 bp, Artificial Sequence
  artificially synthesized recombinant HDM2 (1-118) - NLS/NES biosensor
cctcagactccactgcacaccagccgtgtcctgaaggaggacaaggaacgatgggaggatgtcaaggaggagatgaccagtgccttggccacgatgtgtgttgactatgagcagatcaagataaagaagatagaagacgcatccaaccctctgcttctcaagaggcggaagaaactcgagatgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcataa

SEQ ID NO: 23 - protein, 178 aa, Artificial Sequence
  artificially synthesized recombinant HDM2 (1-118) - NLS/NES biosensor
PQTPLHTSRVLKEDKERWEDVKEEMTSALATMCVDYEQIKIKKIEDASNPLLLKRRKKLEMCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDS

SEQ ID NO: 24 - DNA, 1545 bp, Artificial Sequence
  artificially synthesized recombinant TagRFP-NES/NLS-p25 biosensor
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaattccggactcagatctcgagcccctcagactccactgcacaccagccgtgtcctgaaggaggacaaggaacgatgggaggatgtcaaggaggagatgaccagtgccttggccacgatgtgtgttgactatgagcagatcaagataaagaagatagaagacgcatccaaccctctgcttctcaagaggcggaagaaatcgaattccgcccagcccccaccggcccagccgcctgcacccccggccagccagctctcgggttcccagaccgggggctcctcctcagtcaagaaagcccctcaccctgccgtcacctccgcagggacgcccaaacgggtcatcgtccaggcgtccaccagtgagctgcttcgctgcctgggtgagtttctctgccgccggtgctaccgcctgaagcacctgtcccccacggaccccgtgctctggctgcgcagcgtggaccgctcgctgcttctgcagggctggcaggaccagggcttcatcacgccggccaacgtggtcttcctctacatgctctgcagggatgttatctcctccgaggtgggctcggatcacgagctccaggccgtcctgctgacatgcctgtacctctcctactcctacatgggcaacgagatctcctacccgctcaagcccttcctggtggagagctgcaaggaggccttttgggaccgttgcctctctgtcatcaacctcatgagctcaaagatgctgcagataaatgccgacccacactacttcacacaggtcttctccgacctgaagaacgagagcggccaggaggacaagaagcggctcctcctaggcctggatcggtga

SEQ ID NO: 25 - protein, 514 aa, Artificial Sequence
  artificially synthesized recombinant TagRFP-NES/NLS-p25 biosensor
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRSDMALKLVGGGHLICNFKTTYRSKKPAKNLKMPGVYYVDHRLERIKEADKETYVEQHEVAVARYCDLPSKLGHKLNSGLRSRAPQTPLHTSRVLKEDKERWEDVKEEMTSALATMCVDYEQIKIKKIEDASNPLLLKRRKKSNSAQPPPAQPPAPPASQLSGSQTGGSSSVKKAPHPAVTSAGTPKRVIVQASTSELLRCLGEFLCRRCYRLKHLSPTDPVLWLRSVDRSLLLQGWQDQGFITPANVVFLYMLCRDVISSEVGSDHELQAVLLTCLYLSYSYMGNEISYPLKPFLVESCKEAFWDRCLSVINLMSSKMLQINADPHYFTQVFSDLKNESGQEDKKRLLLGLDR

SEQ ID NO: 26 - protein, 209 aa, Artificial Sequence
  artificially synthesized recombinant p25
AQPPPAQPPAPPASQLSGSQTGGSSSVKKAPHPAVTSAGTPKRVIVQASTSELLRCLGEFLCRRCYRLKHLSPTDPVLWLRSVDRSLLLQGWQDQGFITPANVVFLYMLCRDVISSEVGSDHELQAVLLTCLYLSYSYMGNEISYPLKPFLVESCKEAFWDRCLSVINLMSSKMLQINADPHYFTQVFSDLKNESGQEDKKRLLLGLDR

SEQ ID NO: 27 - DNA, 1377 bp, Artificial Sequence
  artificially synthesized recombinant TagRFP-p25 biosensor
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaattccggactcagatctcgagctcaagcttcgaattccgcccagcccccaccggcccagccgcctgcacccccggccagccagctctcgggttcccagaccgggggctcctcctcagtcaagaaagcccctcaccctgccgtcacctccgcagggacgcccaaacgggtcatcgtccaggcgtccaccagtgagctgcttcgctgcctgggtgagtttctctgccgccggtgctaccgcctgaagcacctgtcccccacggaccccgtgctctggctgcgcagcgtggaccgctcgctgcttctgcagggctggcaggaccagggcttcatcacgccggccaacgtggtcttcctctacatgctctgcagggatgttatctcctccgaggtgggctcggatcacgagctccaggccgtcctgctgacatgcctgtacctctcctactcctacatgggcaacgagatctcctacccgctcaagcccttcctggtggagagctgcaaggaggccttttgggaccgttgcctctctgtcatcaacctcatgagctcaaagatgctgcagataaatgccgacccacactacttcacacaggtcttctccgacctgaagaacgagagcggccaggaggacaagaagcggctcctcctaggcctggatcggtga

SEQ ID NO: 28 - protein, 458 aa, Artificial Sequence
  artificially synthesized recombinant TagRFP-p25 biosensor
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRSDMALKLVGGGHLICNFKTTYRSKKPAKNLKMPGVYYVDHRLERIKEADKETYVEQHEVAVARYCDLPSKLGHKLNSGLRSRAQASNSAQPPPAQPPAPPASQLSGSQTGGSSSVKKAPHPAVTSAGTPKRVIVQASTSELLRCLGEFLCRRCYRLKHLSPTDPVLWLRSVDRSLLLQGWQDQGFITPANVVFLYMLCRDVISSEVGSDHELQAVLLTCLYLSYSYMGNEISYPLKPFLVESCKEAFWDRCLSVINLMSSKMLQINADPHYFTQVFSDLKNESGQEDKKRLLLGLDR

SEQ ID NO: 29 - DNA, 1671 bp, Artificial Sequence
  artificially synthesized recombinant TagRFP-p35 biosensor
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaattccggactcagatctcgagctcaagcttcgaattccatgggcacggtgctgtccctgtctcccagctaccggaaggccacgctgtttgaggatggcgcggccaccgtgggccactatacggccgtacagaacagcaagaacgccaaggacaagaacctgaagcgccactccatcatctccgtgctgccttggaagagaatcgtggccgtgtcggccaagaagaagaactccaagaaggtgcagcccaacagcagctaccagaacaacatcacgcacctcaacaatgagaacctgaagaagtcgctgtcgtgcgccaacctgtccacattcgcccagcccccaccggcccagccgcctgcacccccggccagccagctctcgggttcccagaccgggggctcctcctcagtcaagaaagcccctcaccctgccgtcacctccgcagggacgcccaaacgggtcatcgtccaggcgtccaccagtgagctgcttcgctgcctgggtgagtttctctgccgccggtgctaccgcctgaagcacctgtcccccacggaccccgtgctctggctgcgcagcgtggaccgctcgctgcttctgcagggctggcaggaccagggcttcatcacgccggccaacgtggtcttcctctacatgctctgcagggatgttatctcctccgaggtgggctcggatcacgagctccaggccgtcctgctgacatgcctgtacctctcctactcctacatgggcaacgagatctcctacccgctcaagcccttcctggtggagagctgcaaggaggccttttgggaccgttgcctctctgtcatcaacctcatgagctcaaagatgctgcagataaatgccgacccacactacttcacacaggtcttctccgacctgaagaacgagagcggccaggaggacaagaagcggctcctcctaggcctggatcggtga

SEQ ID NO: 30 - protein, 556 aa, Artificial Sequence
  artificially synthesized recombinant TagRFP-p35 biosensor
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRSDMALKLVGGGHLICNFKTTYRSKKPAKNLKMPGVYYVDHRLERIKEADKETYVEQHEVAVARYCDLPSKLGHKLNSGLRSRAQASNSMGTVLSLSPSYRKATLFEDGAATVGHYTAVQNSKNAKDKNLKRHSIISVLPWKRIVAVSAKKKNSKKVQPNSSYQNNITHLNNENLKKSLSCANLSTFAQPPPAQPPAPPASQLSGSQTGGSSSVKKAPHPAVTSAGTPKRVIVQASTSELLRCLGEFLCRRCYRLKHLSPTDPVLWLRSVDRSLLLQGWQDQGFITPANVVFLYMLCRDVISSEVGSDHELQAVLLTCLYLSYSYMGNEISYPLKPFLVESCKEAFWDRCLSVINLMSSKMLQINADPHYFTQVFSDLKNESGQEDKKRLLLGLDR

SEQ ID NO: 31 - DNA, 1146 bp, Artificial Sequence
  artificially synthesized recombinant HA-NES/NLS-p35 biosensor
atgtacccatacgatgttccagattacgctcttgccggccctcagactccactgcacaccagccgtgtcctgaaggaggacaaggaacgatgggaggatgtcaaggaggagatgaccagtgccttggccacgatgtgtgttgactatgagcagatcaagataaagaagatagaagacgcatccaaccctctgcttctcaagaggcggaagaaatcgaattccatgggcacggtgctgtccctgtctcccagctaccggaaggccacgctgtttgaggatggcgcggccaccgtgggccactatacggccgtacagaacagcaagaacgccaaggacaagaacctgaagcgccactccatcatctccgtgctgccttggaagagaatcgtggccgtgtcggccaagaagaagaactccaagaaggtgcagcccaacagcagctaccagaacaacatcacgcacctcaacaatgagaacctgaagaagtcgctgtcgtgcgccaacctgtccacattcgcccagcccccaccggcccagccgcctgcacccccggccagccagctctcgggttcccagaccgggggctcctcctcagtcaagaaagcccctcaccctgccgtcacctccgcagggacgcccaaacgggtcatcgtccaggcgtccaccagtgagctgcttcgctgcctgggtgagtttctctgccgccggtgctaccgcctgaagcacctgtcccccacggaccccgtgctctggctgcgcagcgtggaccgctcgctgcttctgcagggctggcaggaccagggcttcatcacgccggccaacgtggtcttcctctacatgctctgcagggatgttatctcctccgaggtgggctcggatcacgagctccaggccgtcctgctgacatgcctgtacctctcctactcctacatgggcaacgagatctcctacccgctcaagcccttcctggtggagagctgcaaggaggccttttgggaccgttgcctctctgtcatcaacctcatgagctcaaagatgctgcagataaatgccgacccacactacttcacacaggtcttctccgacctgaagaacgagagcggccaggaggacaagaagcggctcctcctaggcctggatcggtga

SEQ ID NO: 32 - protein, 381 aa, Artificial Sequence
  artificially synthesized recombinant HA-NES/NLS-p35 biosensor
MYPYDVPDYALAGPQTPLHTSRVLKEDKERWEDVKEEMTSALATMCVDYEQIKIKKIEDASNPLLLKRRKKSNSMGTVLSLSPSYRKATLFEDGAATVGHYTAVQNSKNAKDKNLKRHSIISVLPWKRIVAVSAKKKNSKKVQPNSSYQNNITHLNNENLKKSLSCANLSTFAQPPPAQPPAPPASQLSGSQTGGSSSVKKAPHPAVTSAGTPKRVIVQASTSELLRCLGEFLCRRCYRLKHLSPTDPVLWLRSVDRSLLLQGWQDQGFITPANVVFLYMLCRDVISSEVGSDHELQAVLLTCLYLSYSYMGNEISYPLKPFLVESCKEAFWDRCLSVINLMSSKMLQINADPHYFTQVFSDLKNESGQEDKKRLLLGLDR

SEQ ID NO: 33 - protein, 10 aa, Artificial Sequence
  artificially synthesized HA epitope
YPYDVPDYAL

SEQ ID NO: 34 - DNA, 852 bp, Artificial Sequence
  artificially synthesized recombinant HA-NES/NLS-p25 biosensor
atgtacccatacgatgttccagattacgctcttgccggccctcagactccactgcacaccagccgtgtcctgaaggaggacaaggaacgatgggaggatgtcaaggaggagatgaccagtgccttggccacgatgtgtgttgactatgagcagatcaagataaagaagatagaagacgcatccaaccctctgcttctcaagaggcggaagaaatcgaattccgcccagcccccaccggcccagccgcctgcacccccggccagccagctctcgggttcccagaccgggggctcctcctcagtcaagaaagcccctcaccctgccgtcacctccgcagggacgcccaaacgggtcatcgtccaggcgtccaccagtgagctgcttcgctgcctgggtgagtttctctgccgccggtgctaccgcctgaagcacctgtcccccacggaccccgtgctctggctgcgcagcgtggaccgctcgctgcttctgcagggctggcaggaccagggcttcatcacgccggccaacgtggtcttcctctacatgctctgcagggatgttatctcctccgaggtgggctcggatcacgagctccaggccgtcctgctgacatgcctgtacctctcctactcctacatgggcaacgagatctcctacccgctcaagcccttcctggtggagagctgcaaggaggccttttgggaccgttgcctctctgtcatcaacctcatgagctcaaagatgctgcagataaatgccgacccacactacttcacacaggtcttctccgacctgaagaacgagagcggccaggaggacaagaagcggctcctcctaggcctggatcggtga

SEQ ID NO: 35 - protein, 283 aa, Artificial Sequence
  artificially synthesized recombinant HA-NES/NLS-p25 biosensor
MYPYDVPDYALAGPQTPLHTSRVLKEDKERWEDVKEEMTSALATMCVDYEQIKIKKIEDASNPLLLKRRKKSNSAQPPPAQPPAPPASQLSGSQTGGSSSVKKAPHPAVTSAGTPKRVIVQASTSELLRCLGEFLCRRCYRLKHLSPTDPVLWLRSVDRSLLLQGWQDQGFITPANVVFLYMLCRDVISSEVGSDHELQAVLLTCLYLSYSYMGNEISYPLKPFLVESCKEAFWDRCLSVINLMSSKMLQINADPHYFTQVFSDLKNESGQEDKKRLLLGLDR

SEQ ID NO: 36 - DNA, 1860 bp, Artificial Sequence
  artificially synthesized recombinant CDK5DN (T33, N144) - rev (1-74) TagGFP biosensor
atgcagaaatacgagaaactggaaaagattggggaaggcacctacggaactgtgttcaaggccaaaaaccgggagactcatgagatcgtggctctgacacgggtgaggctggatgacgatgatgagggtgtgccgagttccgccctccgggagatctgcctactcaaggagctgaagcacaagaacatcgtcaggcttcatgacgtcctgcacagcgacaagaagctgactttggtttttgaattctgtgaccaggacctgaagaagtattttgacagttgcaatggtgacctcgatcctgagattgtaaagtcattcctcttccagctactaaaagggctgggattctgtcatagccgcaatgtgctacacagggacctgaagccccagaacctgctaataaacaggaatggggagctgaaattggctaattttggcctggctcgagcctttgggattcccgtccgctgttactcagctgaggtggtcacactgtggtaccgcccaccggatgtcctctttggggccaagctgtactccacgtccatcgacatgtggtcagccggctgcatctttgcagagctggccaatgctgggcggcctctttttcccggcaatgatgtcgatgaccagttgaagaggatcttccgactgctggggacgcccaccgaggagcagtggccctctatgaccaagctgccagactataagccctatccgatgtacccggccacaacatccctggtgaacgtcgtgcccaaactcaatgccacagggagggatctgctgcagaaccttctgaagtgtaaccctgtccagcgtatctcagcagaagaggccctgcagcacccctacttctccgacttctgtccgcccacgccgtcgacggtacccatggcaggaagaagcggagacagcgacgaagagctcatcagaacagtcagactcatcaagcttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagcccccccgggatccaccggtcgccaccatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatcctcttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatcatgcccgacaaggccaacaacggcctggaggtgaacttcaagacccgccacaacatcgagggcggcggcgtgcagctggccgaccactaccagaccaacgtgcccctgggcgacggccccgtgctgatccccatcaaccactacctgagcactcagaccgccatcagcaaggaccgcaacgaggcccgcgaccacatggtgctcctggagtccttcagcgcctgctgccacacccacggcatggacgagctgtacaggtaa

SEQ ID NO: 37 - protein, 619 aa, Artificial Sequence
  artificially synthesized recombinant CDK5DN (T33, N144) - rev (1-74) TagGFP biosensor
MQKYEKLEKIGEGTYGTVFKAKNRETHEIVALTRVRLDDDDEGVPSSALREICLLKELKHKNIVRLHDVLHSDKKLTLVFEFCDQDLKKYFDSCNGDLDPEIVKSFLFQLLKGLGFCHSRNVLHRDLKPQNLLINRNGELKLANFGLARAFGIPVRCYSAEVVTLWYRPPDVLFGAKLYSTSIDMWSAGCIFAELANAGRPLFPGNDVDDQLKRIFRLLGTPTEEQWPSMTKLPDYKPYPMYPATTSLVNVVPKLNATGRDLLQNLLKCNPVQRISAEEALQHPYFSDFCPPTPSTVPMAGRSGDSDEELIRTVRLIKLLYQSNPPPNPEGTRQARRNRRRRWRERQRQIHSISERILSTYLGRSAEPVPLQPPRDPPVATMSGGEELFAGIVPVLIELDGDVHGHKFSVRGEGEGDADYGKLEIKFICTTGKLPVPWPTLVTTLCYGIQCFARYPEHMKMNDFFKSAMPEGYIQERTILFQDDGKYKTRGEVKFEGDTLVNRIELKGKDFKEDGNILGHKLEYSFNSHNVYIMPDKANNGLEVNFKTRHNIEGGGVQLADHYQTNVPLGDGPVLIPINHYLSTQTAISKDRNEARDHMVLLESFSACCHTHGMDELYR

SEQ ID NO: 38 - protein, 292 aa, Artificial Sequence
  artificially synthesized recombinant CDK5DN (T33, N144)
MQKYEKLEKIGEGTYGTVFKAKNRETHEIVALTRVRLDDDDEGVPSSALREICLLKELKHKNIVRLHDVLHSDKKLTLVFEFCDQDLKKYFDSCNGDLDPEIVKSFLFQLLKGLGFCHSRNVLHRDLKPQNLLINRNGELKLANFGLARAFGIPVRCYSAEVVTLWYRPPDVLFGAKLYSTSIDMWSAGCIFAELANAGRPLFPGNDVDDQLKRIFRLLGTPTEEQWPSMTKLPDYKPYPMYPATTSLVNVVPKLNATGRDLLQNLLKCNPVQRISAEEALQHPYFSDFCPP

SEQ ID NO: 39 - protein, 6 aa, Artificial Sequence
  artificially synthesized recombinant nuclear localization signal sequence
RRKRQK

SEQ ID NO: 40 - protein, 30 aa, Artificial Sequence
  artificially synthesized recombinant nucleolar localization signal
RKRIRTYLKSCRRMKRSGFEMSRPIPSHLT

SEQ ID NO: 41 - protein, 14 aa, Artificial Sequence
  artificially synthesized recombinant nuclear export signal sequence
CIQQQLGQLTLENL

SEQ ID NO: 42 - protein, 11 aa, Artificial Sequence
  artificially synthesized recombinant nuclear export signal sequence
ELALKLAGLDI

SEQ ID NO: 43 - protein, 11 aa, Artificial Sequence
  artificially synthesized recombinant nuclear export signal sequence
LQLPPLERLTL

SEQ ID NO: 44 - protein, 12 aa, Artificial Sequence
  artificially synthesized recombinant nuclear export signal sequence
ALQKKLEELELD

SEQ ID NO: 45 - protein, 12 aa, Artificial Sequence
  artificially synthesized recombinant nuclear export signal sequence
TLWQFLLHLLLD

(19) United States
(12) Patent Application Publication
(10) Pub. No.: US 2010/0112602 A1
(43) Pub. Date: May 6, 2010

(54) Protein-Protein Interaction Biosensors and Methods of Use Thereof

(76) Inventors: Lansing D. Taylor, Pittsburgh, PA (US); Kenneth A. Giuliano, Pittsburgh, PA (US); Daniel Rajadavid Premkumar, Monroeville, PA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/513,755

(22) PCT Filed: Nov. 9, 2007
(86) PCT No.: PCT/US2007/023678

§ 371 Date: Dec. 30, 2009

Related U.S. Application Data
(60) Provisional application No. 60/858,292, filed on Nov. 10, 2006.
(60) Provisional application No. 60/861,195, filed on Nov. 27, 2006.
(60) Provisional application No. 60/994,852, filed on Sep. 21, 2007.

(51) Int. Cl. G01N 33/53; C07K 14/00; C07H 21/02; C12N 15/74; C12N 5/071

(52) U.S. Cl. 435/7.21; 530/300; 536/23.1; 435/320.1; 435/325; 435/7.1

(57) ABSTRACT

The invention provides methods and reagents for identifying an agent, such as by screening a library of agents, that modulates the interaction of two or more polypeptides, the method comprising: introducing into a cell at least a first polypeptide, each comprising a binding domain, wherein the first polypeptide comprises a localization domain of the second polypeptide; and detecting the cellular location of the first polypeptide, the second polypeptide or a combination thereof, wherein a change in the cellular location of the first polypeptide, the second polypeptide or a combination thereof indicates that the agent modulates the interaction of the two or more polypeptides. The invention also provides methods and reagents for identifying the binding domains of one or more polypeptides.

### RELATED APPLICATIONS

This application claims the benefit of U.S. Provisional Application No. 60/858,292, filed on Nov. 10, 2006, U.S. Provisional Application No. 60/861,195, filed on Nov. 27, 2006, and U.S. Provisional Application No. 60/994,852, filed on Sep. 21, 2007.

The entire teachings of the above applications are incorporated herein by reference.

### BACKGROUND OF THE INVENTION

Interactions among molecules such as proteins and their role in regulating overall cellular functions are fundamental to biochemistry. Protein-protein interactions, as well as interactions with other molecules, such as nucleic acids, carbohydrates, and lipids have been recognized as important drug targets. Such interactions can be correlated, directly or indirectly, with a variety of intracellular events, such as signal transduction, metabolism, cell motility, apoptosis, cell cycle regulation, nuclear morphology, cellular DNA content, microtubule-cytoskeleton stability, and histone phosphorylation. But, although protein-protein interactions have long been considered relevant, they are virtually intractable targets for small molecule drug discovery.

Molecular interactions and the effects of drugs or other treatments on such interactions are currently detected by methods such as in vitro assays where the interactions between purified molecular components are directly measured, two-hybrid systems and variants thereof, in vivo assays where a protein-protein interaction is directly sensed and reported (e.g., fluorescence resonance energy transfer (FRET) between two labeled proteins; incorporation of labeled molecules and detection via antibodies), prediction-based approaches where libraries of 3-D protein structures are scanned for potential protein interaction sites based on data sets composed of known protein-protein or protein-ligand interaction structures, and protein tagging and purification or protein-protein complexes followed by mass spectroscopy analysis. These methods, however, have numerous disadvantages. For example, low sensitivity of detection, large time requirements for assays, the need to construct multiple chimeric proteins, the inability to monitor molecular binding and its effects in live cells, and the need for specialized and expensive equipment, are all limitations on current detection methods. Thus, improved reagents and methods for detecting and measuring molecular binding events and their effects on other cellular functions are needed.

Detailed knowledge of the complex topography of protein-protein interaction sites has been helpful in the design of new protein-protein interaction inhibitors. However, the art lacks methods and reagents to decipher the large number of dynamically interacting protein domains that regulate cellular biochemistry, especially within the context of the living cell where these interactions are to be targeted by new drugs. Furthermore, the successful development of small molecule effectors of protein-protein interactions will need to overcome inadequate efficacy due to low affinity and toxicity due to non-specific protein binding (Fry, D. C. and L. T. Vassilev, J Mol Med, 2005. 83(12):955-63).

### SUMMARY OF THE INVENTION

The invention provides methods and reagents for identifying an agent that modulates the interaction of two or more polypeptides. The invention also provides methods and reagents for method for identifying the presence of a binding domain in a polypeptide to be assessed. Also provided are composition comprising at least two polypeptides for screening drugs for treatment of a neurodegenerative disease.

In one aspect of the invention is a method for identifying an (one or more) agent that modulates the interaction of two or more polypeptides. The method comprises introducing into a cell at least a first polypeptide and a second polypeptide, each comprising a binding domain, a localization domain, and a reporter domain, wherein the first polypeptide comprises a localization domain that is different from the localization domain of the second polypeptide. The cell is maintained under conditions in which the binding domain of the first polypeptide interacts with the binding domain of the second polypeptide, which results in co-localization of the first polypeptide and the second polypeptide at a first cellular location in the cell. An agent is introduced to the cell and the cellular location of the first polypeptide, the second polypeptide or a combination thereof is detected, wherein a change in the cellular location of the first polypeptide, the second polypeptide or a combination thereof as compared to the cellular location before introduction of the agent indicates that the agent modulates the interaction of the two or more polypeptides.

In another aspect of the invention is a method for identifying an agent that modulates the interaction of two or more polypeptides, comprising introducing into a cell at least a first polypeptide and a second polypeptide, each comprising a binding domain, a localization domain, and a reporter domain, wherein the first polypeptide comprises a nuclear localization domain and the second polypeptide comprises a nuclear-cytoplasmic shuttling localization domain. The cell is maintained under conditions in which the binding domain of the first polypeptide interacts with the binding domain of the second polypeptide, which results in co-localization of the first polypeptide and the second polypeptide in the nucleus of the cell. An agent is introduced to the cell and the cellular location of the second polypeptide is detected, wherein a change in the cellular location of the second polypeptide from the nucleus of the cell indicates that the agent modulates the interaction of the two or more polypeptides.

Another aspect of the invention is a method for identifying the presence of a binding domain in a polypeptide to be assessed. The method comprises introducing into a cell a first polypeptide comprising a localization domain, a reporter domain, and a binding domain. The polypeptide to be assessed which comprises a reporter domain, and a localization domain that is different from the localization domain of the first polypeptide is also introduced to the cell. The cell is maintained under conditions in which the first polypeptide interacts with the polypeptide to be assessed when the second polypeptide comprises a binding domain that is capable of binding to the binding domain of the first polypeptide. The method further comprises determining the cellular location of the polypeptide to be assessed, such that if the polypeptide to be assessed co-localizes with the first polypeptide, this indicates that the first polypeptide interacts with the polypeptide to be assessed and that a binding domain is present in the polypeptide to be assessed.

A further aspect of the invention is a polypeptide comprising at least a fragment of a neurodegenerative disease-associated protein, wherein the fragment comprises a binding domain, a reporter domain and a localization domain.

Another aspect of the invention is a composition comprising at least two polypeptides for screening drugs for treatment of a neurodegenerative disease, comprising a first polypeptide that comprises at least a fragment of a neurodegenerative disease-associated protein, wherein the fragment comprises a binding domain, a localization domain, and a reporter domain and a second polypeptide that comprises a binding domain, a localization domain, and a reporter domain, wherein the localization domain of the second polypeptide is different from the localization domain of the first polypeptide, and wherein the binding domain of the first polypeptide binds to the binding domain of the second polypeptide.

Also provided herein is a polypeptide comprising, consisting of, or consisting essentially of an amino acid sequence selected from the group consisting of: SEQ ID NOS: 2, 7, 12, 15, 19, 21, 23, 25, 28, 30, 32, 35, and 37.

Furthermore, provided herein is a nucleic acid sequence encoding a sequence selected from the group consisting of: SEQ ID NOS: 2, 7, 12, 15, 19, 21, 23, 25, 28, 30, 32, 35, and 37. Also provided is a nucleic acid sequence comprising, consisting of, or consisting essentially of a sequence selected from the group consisting of SEQ ID NOS: 1, 6, 11, 14, 18, 20, 22, 24, 27, 29, 21, 34, and 36.

In one aspect of the invention is a polypeptide comprising a binding domain, a localization domain, and a reporter domain, wherein the binding domain is selected from the group consisting of: SEQ ID NOS: 5, 10, 13, 17, 26, 38, or a combination thereof.

In another aspect of the invention is a polypeptide comprising a binding domain, a localization domain, and a reporter domain, wherein the localization domain is selected from the group consisting of: SEQ ID NOS: 4, 9, 39, 40, 41, 42, 43, 44, 45, or a combination thereof.

In a further aspect of the invention is a polypeptide comprising a binding domain, a localization domain, and a reporter domain, wherein the reporter domain is selected from the group consisting of: SEQ ID NOS: 3, 8, 16, 33, or a combination thereof.

In a still further aspect of the invention is a polypeptide comprising a binding domain, a localization domain, and a reporter domain, wherein the binding domain is selected from the group consisting of: SEQ ID NOS: 5, 10, 13, 17, 26, 38, or a combination thereof; the localization domain is selected from the group consisting of: SEQ ID NOS: 4, 9, 39, 40, 41, 42, 43, 44, 45, or a combination thereof; and the reporter domain is selected from the group consisting of: SEQ ID NOS: 3, 8, 16, 33, or a combination thereof.

In another aspect of the invention is a vector comprising a nucleic acid sequence encoding a polypeptide, wherein the polypeptide comprises a binding domain, a localization domain, and a reporter domain, wherein the binding domain is selected from the group consisting of: SEQ ID NOS: 5, 10, 13, 17, 26, 38, or a combination thereof; the localization domain is selected from the group consisting of: SEQ ID NOS: 4, 9, 39, 40, 41, 42, 43, 44, 45, or a combination thereof; and the reporter domain is selected from the group consisting of: SEQ ID NOS: 3, 8, 16, 33, or a combination thereof.

Another aspect of the invention is a host cell comprising a vector, wherein the vector comprises a nucleic acid sequence encoding a polypeptide, wherein the polypeptide comprises a binding domain, a localization domain, and a reporter domain, wherein the binding domain is selected from the group consisting of: SEQ ID NOS: 5, 10, 13, 17, 26, 38, or a combination thereof; the localization domain is selected from the group consisting of: SEQ ID NOS: 4, 9, 39, 40, 41, 42, 43, 44, 45, or a combination thereof; and the reporter domain is selected from the group consisting of: SEQ ID NOS: 3, 8, 16, 33, or a combination thereof.

In a further aspect of the invention is a kit comprising (a) a nucleic acid which encodes a polypeptide comprising a binding domain, a localization domain, and a reporter domain, wherein: the binding domain is selected from the group consisting of: SEQ ID NOS: 5, 10, 13, 17, 26, 38, or a combination thereof; the localization domain is selected from the group consisting of: SEQ ID NOS: 4, 9, 39, 40, 41, 42, 43, 44, 45, or a combination thereof; and the reporter domain is selected from the group consisting of: SEQ ID NOS: 3, 8, 16, 33, or a combination thereof; (b) a vector comprising a nucleic acid sequence encoding a polypeptide, wherein the polypeptide comprises a binding domain, a localization domain, and a reporter domain, wherein: the binding domain is selected from the group consisting of: SEQ ID NOS: 5, 10, 13, 17, 26, 38, or a combination thereof; the localization domain is selected from the group consisting of: SEQ ID NOS: 4, 9, 39, 40, 41, 42, 43, 44, 45, or a combination thereof; and the reporter domain is selected from the group consisting of: SEQ ID NOS: 3, 8, 16, 33, or a combination thereof; (c) a host cell comprising a vector, wherein the vector comprises a nucleic acid sequence encoding a polypeptide, wherein the polypeptide comprises a binding domain, a localization domain, and a reporter domain, wherein: the binding domain is selected from the group consisting of: SEQ ID NOS: 5, 10, 13, 17, 26, 38, or a combination thereof; the localization domain is selected from the group consisting of: SEQ ID NOS: 4, 9, 39, 40, 41, 42, 43, 44, 45, or a combination thereof; and the reporter domain is selected from the group consisting of: SEQ ID NOS: 3, 8, 16, 33, or a combination thereof; or any combination of (a), (b) or (c), and further comprising instructions for use.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic of example Cdk5:p35 and Cdk5:p25 protein-protein interaction biosensor (PPIB, also referred to herein as a “biosensor”) designs, which are embodiments of the invention. The biosensors are built as protein pairs (also referred to herein as “biosensor components”), four of which are shown here. For example, in one embodiment, the first pair consists of a nuclear localized enzymatically inactivated Cdk5 (e.g., CDK5 dominant negative “CDK5DN” mutant such as CDK5 T33, N144), which retains its ability to bind p35 and p25, and a nuclear-cytoplasmic shuttling full length p35. The two components are tagged with distinctly colored fluorescent proteins, which enable quantification of the location of each biosensor component within cells. In other examples, enzymatically active CDK5 is incorporated into the biosensor.

FIG. 2 is a schematic model of the protein-protein interaction biosensor mechanism of action. When the two color biosensors, for exemplification purposes such as those described in FIG. 1, are expressed in untreated cells, the two components interact. Thus, the nuclear or nucleolus-anchored component causes the shuttling component to partition strongly in the nucleolus and a measurement of untreated cells provides a cytoplasm/nucleolus ratio <1. In cells where the interaction between the protein pair (e.g., Cdk5 and p35/p25 in this example) is disrupted with a drug, the shuttling component biosensor is free to re-partition predominately into the cytoplasm. A measurement of cells treated with a disruptor of the specific protein-protein interaction provides a cytoplasm/nucleolus ratio >1.

FIG. 3 are photographs of cells illustrating the characterization of a pair of Cdk5:p35 protein-protein interaction biosensor components expressed individually. U2OS osteosarcoma cells were nucleofected with vectors expressing either a green (TagGFP) nuclear localized Cdk5 component (left panels) or a red (TagRFP) nuclear-cytoplasmic shuttling p35 component (right panels). The Cdk5 biosensor component showed a dominant nuclear location and the p35 biosensor component exhibited a nuclear-cytoplasmic distribution. Thus, when expressed individually, the biosensor components displayed the expected functionality.

FIG. 4 are photographs of cells illustrating the characterization of the interaction between a pair of Cdk5:p35 protein-protein interaction biosensor components co-expressed in cells. U2OS cells were nucleofected with vectors encoding green Cdk5 and red p35 biosensor components at three ratios. In each case, both biosensor components showed a biased nuclear location (compare the bottom two panels in each column). The biased partitioning of both biosensor components into the nuclear compartment is consistent with a strong interaction between the biosensor components. A disruptor of the Cdk5:p35 interaction is predicted to induce the measurable change in the distribution of the shuttling p35 biosensor component.

FIG. 5 illustrates the use of cell population distribution maps to further characterize one pair of Cdk5:p35 PPIB components. Quantification of the expression level and distribution of the two biosensor components expressed alone or co-expressed in U2OS cells is shown as a function of the expression level of the green Cdk5 nuclear localized biosensor component. The DNA content of the same population of cells is also shown to provide at least one indication of the effect that the biosensor components may have on normal cell function. Several conclusions were made: 1) The overall expression level of the two biosensor components is greater when they are co-expressed, consistent with their interaction in the nuclear compartment having a buffering effect on the activity of protein complex; 2) The biased nuclear distribution of the biosensor components becomes most homogeneous at higher Cdk5 expression levels; and 3) Cell cycle effects of the biosensor can be detected and can be monitored during the compound screening phase.

FIG. 6 illustrates one embodiment of a Cdk5 biosensor component of a Cdk5:p35 protein-protein interaction biosensor. The nucleotide sequence (SEQ ID NO: 1) and amino acid sequence (SEQ ID NO: 2) are presented for a cdk5-rev-TagGFP biosensor.

FIG. 7 illustrates one embodiment of a p35 biosensor component of a Cdk5:p35 protein-protein interaction biosensor. The nucleotide sequence (SEQ ID NO: 6) and amino acid sequence (SEQ ID NO: 7) are presented for a TagRFP-NES/NLS-p35 biosensor.

FIG. 8 illustrates one embodiment of a p53 biosensor component. The nucleotide sequence (SEQ ID NO: 11) and amino acid sequence (SEQ ID NO: 22) are presented for a GFP-rev-p53 biosensor.

FIG. 9 illustrates a vector map comprising SEQ ID NO: 11.

FIG. 10 illustrates one embodiment of a HDM2 biosensor component. The nucleotide sequence (SEQ ID NO: 14) and amino acid sequence (SEQ ID NO: 15) are presented for a JRED-NES/NLS-HDM2 biosensor.

FIG. 11 illustrates a vector map comprising SEQ ID NO: 13.

FIG. 12 illustrates one embodiment of a HDM2 biosensor component. The nucleotide sequence (SEQ ID NO: 18) and amino acid sequence (SEQ ID NO: 19) are presented for a TagGFP-NES/NLS-HDM2 biosensor.

FIG. 13 illustrates one embodiment of a p53 biosensor component. The nucleotide sequence (SEQ ID NO: 20) and amino acid sequence (SEQ ID NO: 21) are presented for a p53(1-131)-rev(1-74) biosensor.

FIG. 14 illustrates one embodiment of a HDM2 biosensor component. The nucleotide sequence (SEQ ID NO: 22) and amino acid sequence (SEQ ID NO: 23) are presented for a HDM2(1-118)-NLS/NES biosensor.

FIG. 15 is a schematic of a protein-protein interaction biosensor design. A biosensor for the measurement of the intracellular interaction of p53 and HDM2 is shown. The shuttling component of the two-color biosensor encodes the interaction domain of one of the interacting proteins (e.g., HDM2) fused to a fluorescent reporter and a nuclear-cytoplasmic shuttling domain that encode moderately active NLS and NES peptides. This component will be predominately partitioned into the cytoplasmic compartment when the interaction between the two biosensor components is inhibited. The anchored component of the two-color biosensor encodes the interaction domain of the other interacting protein (e.g., p53) fused to a fluorescent reporter and a nucleolar location peptide from the rev-protein that predominately partitions the second biosensor component in the nucleolar compartment, regardless of its interaction with the shuttling component.

FIG. 16 is a schematic of the interaction of biosensors comprising various fragments of human p53 with a cytoplasm-nuclear shuttling HDM2 fragment.

FIG. 17 is a table of the intracellular location of biosensors comprising various fragments of human p53 when expressed alone or with a cytoplasm-nuclear shuttling HDM2 fragment.

FIG. 18 are sample images showing the intracellular location of biosensors comprising various fragments of human p53 when expressed alone or with a cytoplasm-nuclear shuttling HDM2 fragment. The intracellular location of the full length p53 biosensor component was altered as a result of its interaction with the HDM2 fragment biosensor component.

FIG. 19 illustrates one embodiment of a p25 biosensor component. The nucleotide sequence (SEQ ID NO: 24) and amino acid sequence (SEQ ID NO: 25) are presented for a TagRFP-NES/NLS-p25 biosensor.

FIG. 20 illustrates one embodiment of a p25 biosensor component. The nucleotide sequence (SEQ ID NO: 27) and amino acid sequence (SEQ ID NO: 28) are presented for a TagRFP-p25 biosensor.

FIG. 21 illustrates one embodiment of a p35 biosensor component. The nucleotide sequence (SEQ ID NO: 29) and amino acid sequence (SEQ ID NO: 30) are presented for a TagRFP-p35 biosensor.

FIG. 22 illustrates one embodiment of a p35 biosensor component. The nucleotide sequence (SEQ ID NO: 31) and amino acid sequence (SEQ ID NO: 32) are presented for a HA-NES/NLS-p35 biosensor.

FIG. 23 illustrates one embodiment of a p25 biosensor component. The nucleotide sequence (SEQ ID NO: 34) and amino acid sequence (SEQ ID NO: 35) are presented for a HA-NES/NLS-p25 biosensor.

FIG. 24 illustrates one embodiment of a cdk5 kinase-dead biosensor component. The nucleotide sequence (SEQ ID NO: 36) and amino acid sequence (SEQ ID NO: 37) are presented for a CDK5DN(T33, N144)-rev(1-734)-tagGFP biosensor.

FIG. 25 illustrates the detection of the disruption of an intracellular protein-protein interaction using a prototype biosensor for the p53:HDM2 interaction. In untreated U2OS cells expressing the two-component biosensor of p53:HDM2 interaction, the biosensor components are predominately partitioned in the nucleoli (left panel). Upon treatment with the p53:HDM2 disrupting drug nutlin-3, the biosensor rapidly re-partitions predominately to the cytoplasm, consistent with disruption of the p53:HDM2 interaction (right panel).

FIG. 26 illustrates the screening validation for the prototype protein-protein interaction biosensor. A high content screening assay using the prototype biosensor of p53:HDM2 interaction was validated according to industry standards. Example data are shown. Nutlin-3 titration data of quadruplicate samples are shown in the left panel (EC50=1.1 μM) and min/max data from a 384-well microplate are shown in the right panel (Z′=0.86).

FIG. 27 is a table of results for a three day inter-plate validation of the protein-protein interaction biosensor assay. Single min-max plates (192 wells DMSO and 192 wells nutlin-3) were prepared on three consecutive days from three separate biosensor transfection samples. U2OS cells expressing the dual-color biosensor were treated for 2 h with DMSO (0.1%) or 25 μM nutlin-3 before cell fixation and high content screening. Acceptable Z′ values (e.g., >0.5), which have become the industry standard for screen validation, were obtained for each of the three-day samples. Thus, the prototype protein-protein interaction biosensor has been shown to perform as a suitable reagent for high content screening assays.

### DETAILED DESCRIPTION OF THE INVENTION

Several methods exist in the art to determine protein-protein interactions in living cells (Giuliano, K. A., et al., Optimal characteristics of protein-protein interaction biosensors for cellular systems biology profiling, in High Content Screening: Science, Technology, and Applications, S. A. Haney, Editor. 2007, Wiley: New York. p. (in press)). Table I below summarizes these approaches.

TABLE I
Reagents Designed to Detect and Measure Specific
Protein-Protein Interactions In Living Cells

| Reagent | Measurement Technique | Potential Problems |
|---|---|---|
| Fluorescence | Detect increase in FRET by | Over-expression of |
| Resonance Energy | increased acceptor | proteins that alter cell |
| Transfer (FRET) pair | fluorescence and/or donor | functions |
| of fluorescent | quenching. Ratio of | Non-native interactions |
| proteins coupled to | acceptor fluorescence to | Low signal to noise |
| the two targeted | donor fluorescence when | |
| proteins (Wallrabe, | donor excited | |
| H. and A. Periasamy, | | |
| Curr Opin | | |
| Biotechnol, 2005. | | |
| 16(1): 19-27; | | |
| Miyawaki, A., et al., | | |
| Nature, 1997. | | |
| 388: 882-887) | | |
| Fluorescence | The two fluorescent protein | Complementation shows |
| complementation of | fragments fused to the two | time lag |
| two fragments of a | target proteins re-fold to | Complementation is |
| fluorescent protein | create a fluorescent molecule | irreversible |
| fused to two targeted | when the target proteins bind | Over-expression of |
| proteins (Remy, I. | | proteins that alter cell |
| and S. W. Michnick, | | functions |
| Proc Natl Acad Sci, | | Non-native interactions |
| 2001. 98(14): 7678-83; | | |
| Michnick, S. W., | | |
| Drug Discov Today, | | |
| 2004. 9(6): 262-7) | | |
| Luminescence | The two luciferase protein | Complementation shows |
| complementation of | fragments fused to the two | time lag |
| two fragments of | target proteins re-fold to | Complementation is |
| luminescent enzymes | create a luminescent enzyme | irreversible** |
| e.g. luciferase* | when the target proteins bind | Over-expression of |
| (Remy, I. and S. W. | | proteins that alter cell |
| Michnick, Nat | | functions |
| Methods, 2006. | | Non-native interactions |
| 3(12): 977-9; | | Requires addition of |
| Kerppola, T. K., Nat | | coelenterazine for signal |
| Methods, 2006. | | |
| 3(12): 969-71) | | |
| Positional Biosensors | Change in the cellular | Over-expression of |
| (Giuliano, K. A., et | compartment of one of the | proteins that alter cell |
| al., Reagents to | proteins of a pair based on | function |
| measure and | NLS and NES sequences on | Non-native interactions |
| manipulate cell | biosensor | |
| functions, in High | | |
| Content Screening: A | | |
| Powerful Approach | | |
| to Systems Cell | | |
| Biology and Drug | | |
| Discovery, D. L. | | |
| Taylor, Haskins, J. R., | | |
| and Giuliano, K. A., | | |
| Editor. 2006, | | |
| Humana Press: | | |
| Totowa, NJ. p. 141-163) | | |

*Protein complementation assays (PCA's) have been developed based on other enzymes (Kerppola, T. K., Nat Methods, 2006. 3(12): 969-71).
**Indication that a Gaussia luciferase might be reversible (Remy, I. and S. W. Michnick, Nat Methods, 2006. 3(12): 977-9).

In addition, other methods such as yeast two-hybrid, mammalian protein-protein interaction trap (MAPPIT) (Eyckerman, S., et al., Nat Methods, 2005. 2(6):427-33), and the proximity-ligation in situ assay (P-LISA) that are either not as specific or are not applied to living cells, also have shown promise (Lievens, S. and J. Tavernier, Nat Methods, 2006. 3(12):971-2).

Table II below lists optimal characteristics of protein-based biosensors.

TABLE II
Optimal Characteristics of Protein-Based Biosensors
Using Fluorescence or Luminescence for Detection

| Optimal Characteristic | Potential Problem |
|---|---|
| Biosensor present at concentration less | Biosensor concentration overwhelms |
| than native protein (optimally less than | native protein and does not report native |
| 10%) | functions or regulation |
| Biosensor demonstrates at least 90% of | Biosensor does not report desired protein |
| native protein function or at least % | functions or kinetics |
| defined | |
| Biosensor does not alter cell activity by | Presence of biosensor alters cell activity |
| its presence | |
| Biosensor is reversible | Biosensor activation is irreversible |
| | leading to non-native responses |

Reviewing the reagents used to detect and to measure protein-protein interactions in Table I and the optimal characteristics of protein-based biosensors in Table II suggests that the present pairs of fluorescent proteins used for FRET, in general, do not yield a high enough signal to noise ratio for large-scale screening. However, a recent report suggests that an improved pair of fluorescent proteins might improve this characteristic, but probably not enough for screening (You, X., et al., Proc Natl Acad Sci USA, 2006. 103(49):18458-63). Although the optimal traits of FRET include temporal response time of the signal and reversibility, the typical levels of biosensor overexpression used to optimize the signal to noise ratio causes concern about over-whelming the native protein functions. In some cases the biosensors become “modulators” of activity, not reporters. In addition, some of the protein functions might be significantly altered by the labeling. The primary method to determine level of protein function after labeling has usually been “native” localization compared to antibody labeling. However, more functional measurements are useful. In addition, some of the protein functions might be significantly altered by the labeling.

The fluorescence-based complementation reagents have the same issues as the FRET reagents, but there is an additional concern over the lag time required to develop fluorescence during the refolding of the pair of complementation halves. In addition, the refolding of the complementation partners appears to be irreversible. This latter characteristic makes the measurement of any downstream cellular responses questionable. The complementation approach must be improved by making the complementation reversible when the tagged proteins dissociate (Remy, I. and S. W. Michnick, Nat Methods, 2006. 3(12):977-9).

The luminescence version of the complementation reagents have the same issues as the fluorescence-based complementation reagents, but with the added requirement of exogenous coelenterazine to fuel the luminescence signal. A recent report indicates that the complementation of a luciferase from Gaussia is reversible and should replace existing non-reversible luciferase methods in functional studies (Remy, I. and S. W. Michnick, Nat Methods, 2006. 3(12):977-9). In a cellular systems biology profile, there is some question as to the effect of coelenterazine on cell function. Detailed controls on the effect of coelenterazine on a range of cell functions such as cell cycle, metabolism, etc. should be performed.

Described herein are use of protein-protein interaction biosensors (PPIB, also referred to herein as “positional biosensors”, or “positional biosensors of protein-protein interactions”) which have fewer potential problems than the other live cell approaches to protein-protein interactions. Although there is a potential of functional problems induced by overexpression, very low levels of expression can be used, since the change in cellular compartment can be measured with a high Z′ factor. Keeping this percentage low is also useful for optimizing the physiological relevance of the measurements.

Thus, provided herein are methods and reagents that can be used to: 1) determine the binding domains of a large number of interacting proteins under conditions found within living cells; and 2) measure the effects of ions, small molecules, and macromolecules on reversible protein-protein interactions in living cells.

Positional biosensors of protein-protein interactions use the intracellular location of one or more of their components as a readout for a reversible protein-protein interaction. That the PPIB components are reversibly bound to each other enables testing of inhibitory molecules, including macromolecules such as proteins and peptides, nucleic acids such as DNA, RNA, and aptamers, simple and complex carbohydrates, and fatty acids and other lipid molecules, as well as smaller compounds and ions for their ability to prevent or enhance the interaction of the PPIB components.

Specifically provided herein are positional biosensors comprising polypeptides. In one embodiment, the polypeptides are recombinant polypeptides comprising, consisting, or consisting essentially of a binding domain, a localization domain, and a reporter domain. Different biosensors have been described previously, see, e.g., WO2006/017751, the teachings of which are incorporated herein by reference in their entirety.

As used herein, a “binding domain” is a region (e.g., of a polypeptide) that is sufficient to bind to another binding domain in another molecule (e.g., a polypeptide, a biosensor, etc.). The binding domain is a region of a polypeptide to which a molecule interacts. For example, as shown herein, the molecule can be a binding domain present in another polypeptide. The binding domain of a polypeptide for use in the methods of the invention may be a naturally occurring binding domain. In addition, mutants, variants, or fragments of such naturally occurring binding domains, or an artificial domain or recombinant domain, can be used in the methods. The binding domain can comprise more than just a binding domain, e.g., polypeptide sequences that do not comprise a binding domain, or amino acid sequences that flank a binding domain. Alternatively, the binding domain consists essentially of only the polypeptide sequence necessary for binding. Binding may be by covalent or non-covalent interaction. Such binding domains can be a binding domain isolated from known polypeptides, a putative binding domain or recombinantly prepared or artificially synthesized. For example, the binding domain can be a binding domain present in a normal cellular molecule, a disease-associated molecule, a non-disease-associated molecule, a cell cycle associated molecule, a tissue-specific molecule, and the like.

A disease-associated molecule (e.g., a protein) can be a neurodegenerative disease-associated molecule or a cancer-associated molecule. Such molecules are known in the art. In one embodiment, the binding domain comprise all or a portion of the binding domain of p35, p25, cyclin dependent kinase 5 (cdk5), p53, human double minute 2 (HDM2), and the like. Such binding domains may include full-length proteins, or fragments thereof. Such fragments comprise at least a portion of a binding domain of the protein. In one embodiment, the binding domain can comprise a molecule (e.g., a protein or a polypeptide) that has been mutated to change or alter one or more activities of the protein or polypeptide. For example, a binding domain can comprise all or part of a binding domain of a kinase wherein the kinase is a kinase-inactive or kinase-dead mutant. Such mutants can be useful where the activity of the molecule may otherwise be toxic to a cell. In one embodiment, a binding domain comprises all or part of a CDK5 dominant-negative (CDK5DN) mutant. In a particular embodiment, the CDK5DN is a CDK5DN(T33, N 144) mutant.

In one embodiment, the polypeptide comprises at least a fragment of a neurodegenerative disease-associated protein, wherein the fragment comprises a binding domain. A neurodegenerative disease-associated protein is any protein whose expression is associated with a neurodegenerative disease. A neurodegenerative-disease associated protein can be a protein normally found in a cell, but is in abnormal quantities, conformation or location in a diseased cell (e.g., tau), a truncated protein or cleavage product of a normal protein (e.g., p25 which is a cleavage product of the p35), an abnormally hyper- or hypo-phosphorylated protein (e.g., tau, tyrosine kinase receptors such as the insulin receptor, and DNA interacting proteins such as histones, and the like. The disease can be, e.g., Alzheimer's disease, amyotrophic lateral sclerosis, ataxia telangiectasia, Creutzfeldt-Jakob disease, Huntington disease, multiple sclerosis, Parkinson disease, primary lateral sclerosis, and the like. Neurodegenerative disease-associated proteins are known in the art, and include tau, p25/cdk5, etc. In one embodiment, the neurodegenerative disease-associated protein is p25.

In another embodiment, the polypeptide comprises at least a fragment of a cancer-associated protein, wherein the fragment comprises a binding domain. A cancer-associated protein is any protein whose expression is associated with a cancer. cancer associated proteins are known in the art, and includes p53.

As described herein, a polypeptide of the invention comprises a localization domain. As used herein, a “localization domain” includes a region of polypeptide sequence that provides a selection for cellular distribution (directs the cellular localization of the polypeptide to which it is attached) of the polypeptide to one or more particular cellular locations or subcellular compartments of the cell. As used herein, a “cellular location” refers to any structural or sub-structural macromolecular component of the cell, whether it is made of protein, lipid, carbohydrate, or nucleic acid. For example, a cellular location can be a macromolecular assembly or an organelle (a membrane delineated cellular compartment). Cellular locations include, but are not limited to locations such as cytoplasm, nucleus, nucleolus, the nuclear envelope, regions within the nucleus with localized activities such as transcription, cytoskeleton, inner membrane (e.g., plasma, nuclear), outer plasma membrane, (e.g., plasma) mitochondrial membrane, inner mitochondria, Golgi, endoplasmic reticulum, lysosomes, endocytic vesicles, and extracellular space. In one embodiment, the localization domain of a first polypeptide and a second polypeptide are independently selected from the group consisting of a nuclear localization domain, a nucleolar localization domain, a cytoplasmic localization domain, an organellar localization domain (such as a mitochondrial, peroxisomal and/or centrosomal), and a combination thereof. In one embodiment, the localization domains of two or more polypeptides as described herein, are different from each other.

For example, the localization domain of one polypeptide is a nuclear localization domain and its target location is the nucleus and the localization domain of the other polypeptide is a cytoplasmic localization domain and its target location is the cytoplasm. Alternatively, the localization domain of the first polypeptide directs the location of the first polypeptide to a particular area of the nucleus (e.g., nucleolus) and the localization domain of the other polypeptide is in a different area (location, locale) of the nucleus (e.g., the nuclear membrane). In this embodiment the location and of the two polypeptides when in the nucleus can be distinguished (detected).

When the two or more polypeptides of the invention, which each comprise a different localization domain, interact with each other (e.g., bind to each other via their binding domains), the location of the two or more polypeptides will depend on the relative strengths of the localization domains of each polypeptide (e.g., one localization domain will predominate over the location of the other (one or more) interacting polypeptide(s) in a cell). Such localization domains are known to those of skill in the art and can be isolated, recombinantly prepared or artificially synthesized using standard techniques. For example, a nuclear localization sequence (NLS) domain can comprise all or a portion of the HIV protein rev, all or a portion of the nuclear localization sequence of SV40, the nuclear localization domain RRKRQK (SEQ ID NO: 39) of NFkB p50 (Henkel et al., Cell (1992) 68,1121-1133), the nucleolar localization domain KRIRTYLKSCRRMKRSGFEMSRPIPSHLT (SEQ ID NO: 40) (Ueki, et al., Biochem Biophys Res Commun. (1998) 252:97-102, 1998), and the like. Other localization domains are known in the art, see e.g., U.S. Pat. No. 7,244,614, the teachings of which are incorporated herein by reference in their entirety.

Nuclear export sequences (NES) can comprise the nuclear export sequence of mitogen-activated protein kinase-activated protein kinase 2 (MAPKAP2), Annexin II, IkB-alpha (e.g., CIQQQLGQLTLENL (SEQ ID NO: 41), Jans et al., BioEssays (2000) 22:532-544), PKI-alpha (e.g., ELALKLAGLDI (SEQ ID NO: 42), Jans et al., BioEssays (2000) 22:532-544), HIV Rev (e.g., LQLPPLERLTL (SEQ ID NO: 43), Jans et al., BioEssays (2000) 22:532-544), MAPKK (e.g., ALQKKLEELELD (SEQ ID NO: 44), Jans et al., BioEssays (2000) 22:532-544), hNet (e.g., TLWQFLLHLLLD (SEQ ID NO: 45), Ducret et al., Mol. Cell Biol. (1999) 19:7076-7087), and the like.

Combination NES/NLS localization domains are also known in the art and shuttle the polypeptide to which the localization domain is attached between the cytoplasm and nucleus.

In one embodiment, the localization domain of a first polypeptide is a nuclear localization domain and the localization domain of a second polypeptide is a nuclear export sequence/nuclear-cytoplasmic shuttling localization domain.

As described herein, a polypeptide of the invention comprises a reporter domain. As known to those of skill in the art, a reporter domain provides a means to detect, assess, evaluate the polypeptide in a cell, e.g., the location of a polypeptide in a cell. In one embodiment, the reporter domain of a first polypeptide and the reporter domain of a second polypeptide are the same or different. The reporter domain can comprise any suitable reporter domain known to those of skill in the art. For example, a suitable reporter domain can be a fluorescent protein (e.g., BFP, GFP, RFP) or a tag (e.g., SNAP tag, Halo tag, Lumio tag, a FlAsH tag, an epitope tags (e.g., HA, myc, flag, etc.)), or a combination thereof. A reporter domain can be evaluated (e.g., detected, quantified, localized such as within a cell) using standard techniques, such as detection of fluorescence or luminescence, including detection of fluorescence resonance energy transfer (FRET), fluorescence anisotropy, fluorescence rotational difference, fluorescence lifetime change, fluorescence solvent sensitivity, fluorescence quenching, bioluminescence, chemiluminescence, and the like.

In another embodiment, the polypeptide biosensor comprises, consists of or consists essentially of an amino acid sequence selected from SEQ ID NOS: 2, 7, 12, 15, 19, 21, 23, 25, 28, 30, 32, 35, and 37.

In one embodiment, the polypeptide biosensor comprises a binding domain, a localization domain, and a reporter domain, wherein the binding domain is selected from the group consisting of: SEQ ID NOS: 5, 10, 13, 17, 26, and 38.

In another embodiment, the polypeptide biosensor comprises a binding domain, a localization domain, and a reporter domain, wherein the localization domain is selected from the group consisting of: SEQ ID NOS: 4, 9, 39, 40, 41, 42, 43, 44, and 45.

In another embodiment, the polypeptide biosensor comprises a binding domain, a localization domain, and a reporter domain, wherein the reporter domain is selected from the group consisting of: SEQ ID NOS: 3, 8, 16, and 33.

In a further embodiment, the polypeptide biosensor comprises a binding domain, a localization domain, and a reporter domain, wherein the binding domain is selected from the group consisting of: SEQ ID NOS: 5, 10, 13, 17, 26, and 38; the localization domain is selected from the group consisting of: SEQ ID NOS: 4, 9, 39, 40, 41, 42, 43, 44, and 45; and the reporter domain is selected from the group consisting of: SEQ ID NOS: 3, 8, 16, and 33.

Also provided herein are nucleic acid sequences encoding a biosensor of the present invention. Such nucleic acid sequences can be prepared recombinantly using techniques that are routine in the art. One embodiment of the invention is a nucleic acid sequence comprising, consisting essentially of, or consisting of a sequence selected from: SEQ ID NOS: 1, 6, 11, 14, 18, 20, 22, 24, 27, 29, 21, 34, and 36.

Also provided are vectors, such as expression vectors, comprising the nucleic acid sequences encoding one or more polypeptides of the invention. Vectors can be any construct suitable for bacterial, viral, insect or mammalian propagation and/or expression, as known in the art. Host cells comprising such vectors are also provided by the present invention.

Introduction of one or more polypeptides, or an agent of interest, to a cell can be by any suitable means. As used herein, “introduction to a cell” means both the intracellular incorporation or uptake of the polypeptide or agent into the cell, or the extracellular exposure of a cell to an agent or a polypeptide (e.g., a ligand that binds to a receptor on the surface of the cell such as a tyrosine kinase receptor ligand) as described herein. For example, introduction into a cell can be by transfection, electroporation, optoinjection, membrane translocating signal sequence attachment, cell scraping, detergent treatment of the cell, or other bulk-loading methods. Such methods are standard in the art. Extracellular exposure of a cell to an agent or a polypeptide as described herein can be by adding the agent or polypeptide to the extracellular environment of the cell (e.g., cell culture medium). In particular, the methods and reagents of the invention can be performed or used in living cells, such as vertebrate cells, including mammalian cells (e.g., human cells, rat cells, mouse cells, primate cells and the like), and invertebrate cells (e.g., insect cells and the like). Such cells can be primary cells, stem cells, immortalized cells, cell lines and the like.

The invention described herein provides methods for identifying an agent that modulates the interaction of two or more polypeptides as described above. An (one or more) agent can be any test compound or molecule of interest, such as a drug. In one embodiment, the agent is one or more agents from a library of agents. In another embodiment, the library of agents is a library of macromolecules, small molecules or a combination thereof. As used herein, a small molecule is a small organic molecule of <1000 M.W. Macromolecules are molecules having a >1000 M.W. In one embodiment, a macromolecule is a protein, peptide, nucleic acid (e.g., DNA, RNA, PNA and/or aptamers), simple carbohydrate, complex carbohydrate, fatty acid, lipid molecule, or a combination thereof. Additionally, in one embodiment, the agent can be labeled with a cellular transport peptide, a fluorescent label, or a combination thereof.

Although two polypeptides are typically discussed herein, it is apparent to one of skill in the art that additional polypeptide (e.g., a third, a fourth, etc.) comprising a reporter domain, a localization domain and a binding domain can also be used in the methods described herein. It will also be apparent to one of skill in the art that one or more of the steps of the methods described herein can be performed sequentially or simultaneously.

The method for identifying an agent that modulates the interaction of two or more polypeptides comprises introducing to a cell at least a first polypeptide and a second polypeptide. Both the first polypeptide and the second polypeptide each comprise a binding domain, a localization domain, and a reporter domain, as described above. In one embodiment, the first polypeptide comprises a localization domain that is different from the localization domain of the second polypeptide. The method further comprises maintaining the cell under conditions in which the binding domain of the first polypeptide interacts with the binding domain of the second polypeptide in the cell, which results in co-localization of the first polypeptide and the second polypeptide at a first cellular location in a cell. As one of skill in the art will understand, one binding domain “interacts with” another binding domain by e.g., covalent, non covalent binding.

Conditions under which the cell is maintained so that the binding domain of the first interacts with the binding domain of the second most often typical cell culture conditions as routinely used in the art. See for example, Basic Techniques for Mammalian Tissue Culture, Mary C. Phelan, 2003, Juan S., Bonifacino, et al. (eds.); Current Protocols in Cell Biology, John Wiley & Sons, Inc.

As used herein, “co-localization” refers to the localization of both the first polypeptide and second polypeptide in the same cellular location due to the first polypeptide and second polypeptide interacting via their respective binding domains. In one embodiment, the first polypeptide and second polypeptide co-localize in the cell due to the interaction of the binding domain of the first polypeptide with the binding domain of the second polypeptide, where the localization domain of first polypeptide dominates over the localization domain of the second polypeptide, or vice versa. The cellular location of the co-localizing first polypeptide and second polypeptide can be regulated by the relative strengths of the localization domains to anchor in a particular cellular location.

The method further comprises introducing to the cell an agent, and detecting the cellular location of the first polypeptide, the second polypeptide or a combination thereof, wherein a change in location of the first polypeptide, the second polypeptide or combination thereof as compared to a suitable control, e.g., the cellular location of the first polypeptide, the second polypeptide or a combination thereof, before introducing the agent, indicates that the agent modulates the interaction of the two or more polypeptides. In one embodiment, the agent disrupts the interaction of the two or more polypeptides, thereby permitting one or more polypeptides to change its cellular location in the cell as determined by the localization domain on the one or more polypeptides. In another embodiment, detecting the cellular location of the first polypeptide, the second polypeptide or a combination thereof is performed in the presence of the agent. In another embodiment, detecting the cellular location of the first polypeptide, the second polypeptide or a combination thereof is performed after introduction and subsequent removal of the agent.

In a particular embodiment, the invention is a method for identifying an agent that modulates the interaction of two or more polypeptides, comprising introducing into a cell at least a first polypeptide and a second polypeptide. The first polypeptide and the second polypeptide each comprise a binding domain, a localization domain, and a reporter domain as described above. In a particular embodiment, the first polypeptide comprises a nuclear localization domain and the second polypeptide comprises a nuclear-cytoplasmic shuttling localization domain. The method further comprises maintaining the cell under conditions in which the binding domain of the first polypeptide interacts with the binding domain of the second polypeptide in the cell, which results in co-localization of the first polypeptide and the second polypeptide in the nucleus of the cell. An agent, as described, above can be introduced to the cell and the cellular location of the second polypeptide is determined, wherein a change in location indicates that the agent modulates the interaction of the two or more polypeptides. In one embodiment, the change in location of the second polypeptide is from a nuclear location to a cytoplasmic location. In one embodiment, the binding domain of the first polypeptide comprises all or a portion of a binding domain of cyclin dependent kinase 5 (cdk5) and the binding domain of the second polypeptide comprises all or a portion of a binding domain of p35 or the binding domain of the second polypeptide comprises all or a portion of a binding domain of p25. In another embodiment, the binding domain of the first polypeptide comprises all or a portion of a binding domain of p53 and the binding domain of the second polypeptide comprises all or a portion of a binding domain of HDM2.

Also provided herein is a method for identifying the presence of a binding domain in a polypeptide to be assessed. The method comprises introducing into a cell a first polypeptide comprising a localization domain, a reporter domain, and a binding domain. In a particular embodiment, all or a portion of the binding domain of the first polypeptide is known. Thus, the polypeptide can also be referred to as e.g., a reference polypeptide or an indicator polypeptide. The method further comprises introducing into the cell a (one or more) polypeptide to be assessed (e.g., a second polypeptide; third polypeptide). The polypeptide to be assessed comprises a reporter domain, and a localization domain that is distinct e.g., different, from the localization domain of the first polypeptide. The cell is maintained under conditions in which the first polypeptide interacts with the second polypeptide when the second polypeptide comprises a binding domain that is capable of binding to the binding domain of the first polypeptide. As discussed above, such conditions are typically routine cell culture conditions. The cellular location of the polypeptide being assessed is determined (e.g., detected), wherein if the polypeptide being assessed co-localizes with the first polypeptide (e.g., the polypeptide being assessed does not localize to the cellular location that is inherent to (dictated by) the localization domain of the polypeptide being assessed; the polypeptide being assessed does not localize to the normal cell location of the localization domain of the polypeptide being assessed), this indicates that the first polypeptide interacts with the polypeptide being assessed and that a binding domain is present in the polypeptide being assessed.

In one embodiment, the polypeptide to be assessed for the presence of a binding domain is all or a biologically active portion (e.g. at least a fragment) of an endogenous molecule. As used herein, an “endogenous molecule” is any molecule that is normally found in the cell. In another embodiment, the polypeptide to be assessed for the presence of a binding domain is all or a biologically active portion (e.g. at least a fragment) of an exogenous molecule. As used herein, an “exogenous molecule” is any molecule that is not normally found in the cell, for example a molecule found in a different cell, an artificial molecule, a synthesized molecule, a disease-associated molecule, and the like. A “biologically active portion” is that portion of the polypeptide that can still interact (bind) with a binding domain.

In addition, the invention also provides a composition comprising at least two polypeptides for screening drugs for treatment of a neurodegenerative disease, comprising a first polypeptide comprising a binding domain of a neurodegenerative disease-associated protein, a localization domain, and a reporter domain, and a second polypeptide comprising a binding domain, a localization domain, and a reporter domain, wherein the localization domain of the second polypeptide is different from the localization domain of the first polypeptide, and wherein the binding domain of the first polypeptide binds to the binding domain of the second polypeptide. The second polypeptide can comprise a binding domain of a second neurodegenerative disease-associated protein, or a non-disease-associated protein (e.g., a normal protein). In one embodiment, the first polypeptide comprises all or a portion of a binding domain of p35 or p25, and the second polypeptide comprises all or a portion of a binding domain of cyclin dependent kinase 5 (cdk5).

The invention also comprises a method for screening drugs for treatment of a neurodegenerative disease comprising introducing a first polypeptide comprising a binding domain of a neurodegenerative disease-associated protein, a localization domain, and a reporter domain, and a second polypeptide comprising a binding domain, a localization domain, and a reporter domain, wherein the localization domain of the second polypeptide is different from the localization domain of the first polypeptide, and wherein the binding domain of the first polypeptide binds to the binding domain of the second polypeptide, into a cell. The cell is maintained under conditions in which the binding domain of the first polypeptide interacts with the binding domain of the second polypeptide, which results in co-localization of the first polypeptide and the second polypeptide at a first cellular location in a cell. The method further comprises introducing to the cell one or more drugs to be screened and detecting the cellular location of the first polypeptide, the second polypeptide, or a combination thereof, wherein a change in the cellular location of the first polypeptide, the second polypeptide, or a combination thereof as compared with the cellular location before introduction of the drug, indicates that the agent modulates the interaction of the first polypeptide and second polypeptide and is a candidate drug for the treatment of a neurodegenerative disease. An agent that modulates the interaction of the first polypeptide and second polypeptide can disrupt, enhance or otherwise alter the binding of the first polypeptide to the second polypeptide.

The invention also comprises a method for screening drugs for treatment of a cancer comprising introducing a first polypeptide comprising a binding domain of a cancer-associated protein, a localization domain, and a reporter domain, and a second polypeptide comprising a binding domain, a localization domain, and a reporter domain, wherein the localization domain of the second polypeptide is different from the localization domain of the first polypeptide, and wherein the binding domain of the first polypeptide binds to the binding domain of the second polypeptide into a cell. The second polypeptide can comprise a binding domain of a second cancer-associated protein, or a non-cancer-associated protein (e.g., a normal protein). The cell is maintained under conditions in which the binding domain of the first polypeptide interacts with the binding domain of the second polypeptide, which results in co-localization of the first polypeptide and the second polypeptide at a first cellular location in a cell. The method further comprises introducing to the cell one or more drugs to be screened and detecting the cellular location of the first polypeptide, the second polypeptide, or a combination thereof, wherein a change in the cellular location of the first polypeptide, the second polypeptide, or a combination thereof as compared with the cellular location before introduction of the drug, indicates that the agent modulates the interaction of the first polypeptide and second polypeptide and is a candidate drug for the treatment of a cancer. An agent that modulates the interaction of the first polypeptide and second polypeptide can disrupt, enhance or otherwise alter the binding of the first polypeptide to the second polypeptide. In one embodiment, the first polypeptide comprises all or a portion of a binding domain of p53. In another embodiment, the second polypeptide comprises all or a portion of a binding domain of HDM2.

In another aspect, the invention provides kits comprising a combination of one or more polypeptides of the invention, a nucleic acid sequence encoding one or more polypeptides of the invention, an expression vector comprising one or more nucleic acid sequences encoding one or more polypeptides of the invention, host cells comprising such vectors and instructions for their use in the methods of the invention described herein. In one embodiment, a kit comprises (a) a nucleic acid which encodes a polypeptide comprising a binding domain, a localization domain, and a reporter domain, wherein the binding domain is selected from the group consisting of: SEQ ID NOS: 5, 10, 13, 17, 26, 38, and combinations thereof; the localization domain is selected from the group consisting of: SEQ ID NOS: 4, 9, 39, 40, 41, 42, 43, 44, 45, and combinations thereof; and the reporter domain is selected from the group consisting of: SEQ ID NOS: 3, 8, 16, 33, and combinations thereof; (b) a vector comprising a nucleic acid sequence encoding a polypeptide, wherein the polypeptide comprises a binding domain, a localization domain, and a reporter domain, wherein the binding domain is selected from the group consisting of: SEQ ID NOS: 5, 10, 13, 17, 26, 38, and combinations thereof; the localization domain is selected from the group consisting of: SEQ ID NOS: 4, 9, 39, 40, 41, 42, 43, 44, 45, and combinations thereof; and the reporter domain is selected from the group consisting of: SEQ ID NOS: 3, 8, 16, 33, and combinations thereof; (c) a host cell comprising a vector, wherein the vector comprises a nucleic acid sequence encoding a polypeptide, wherein the polypeptide comprises a binding domain, a localization domain, and a reporter domain, wherein the binding domain is selected from the group consisting of: SEQ ID NOS: 5, 10, 13, 17, 26, 38, and combinations thereof; the localization domain is selected from the group consisting of: SEQ ID NOS: 4, 9, 39, 40, 41, 42, 43, 44, 45, and combinations thereof; and the reporter domain is selected from the group consisting of: SEQ ID NOS: 3, 8, 16, 33, and combinations thereof; or any combination of (a), (b), and (c), the kit further comprising instructions for use.

### EXEMPLIFICATION

Example 1 discloses how a p53-HDM2 PPIB is used to test for peptides that disrupt protein complex formation. Example 2 discloses how a CdkS-p35 PPIB is used to test for aptamers that disrupt protein complex formation. Example 3 discloses a specific PPIB for measurement of the interaction of the kinase Cdk5 with its target proteins p35 and p25 in living. Thus, the invention discloses how multiple classes of molecules can be used to dissect the interaction site between PPIB components, thus enabling users to screen one or more potential drugs for protein-protein interaction modulating activity using specific complexes comprised of two or more proteins or fragments thereof. Furthermore, the invention can be used to produce a molecular template against which new modulators of protein-protein interactions can be designed. In yet another embodiment of the invention, PPIB components are built as fragments of at least two test proteins and used to measure the affinity of the fragments to each other in living cells thus enabling the dissection of the interaction site between two proteins (Example 4).

### Example 1

Testing Inhibitory Peptides of the p53-HDM2 Protein-Protein Interaction

A PPIB of the p53-HDM2 interaction is produced where the components encode portions of p53 (amino acids 1-131) and HDM2 (amino acids 1-118), for example. In one embodiment, the HDM2 component (e.g., nuclear-cytoplasmic shuttling component) encodes a fused TagRFP. Vectors encoding both PPIB components are introduced into cells through transfection, infection with viral expression systems, or other methods. The expressed proteins are allowed to interact. The interaction is measured by the predominant nuclear location of both PPIB components. A set of test inhibitory peptides encoding fragments of either p53 or HDM2 ranging in size from two amino acids to 100 amino acids are synthesized either chemically or produced recombinantly and modified to contain either or both a cellular transport peptide (e.g., antennapedia protein fragment) and a fluorescent label (e.g., fluorescein, rhodamine, GFP, etc.). Cells expressing the PPIB components are then treated with at least one of the inhibitory peptides for a period of time ranging from 1 min to 24 h. Immediately after treatment with the test peptides, the intracellular distribution of both the test peptide and the shuttling HDM2 PPIB component is measured over time either kinetically or using a fixed end point approach. If the peptide inhibits the interaction of the PPIB components, then the shuttling HDM2 biosensor component will distribute predominately to the cytoplasm and the inhibitory peptide will distribute predominately with the PPIB component to which it is most strongly bound.

### Example 2

Testing Inhibitory RNA Aptamers of the p35-Cdk5 Protein-Protein Interaction

A PPIB of the p35-Cdk5 interaction is produced as described where the components encode full length wild type p35 and Cdk5. In this embodiment, the p35 component (e.g., nuclear-cytoplasmic shuttling component) also encodes a fused TagRFP marker. In another embodiment, other labels such as epitopes or other label-binding amino acid sequences can be used as detection domains for the biosensor. The nuclear-anchored Cdk5 component of the PPIB optionally also encodes a fused TagGFP marker. Cells are transfected with vectors encoding both PPIB components. The expressed proteins are allowed to interact. The interaction is measured by the predominant nuclear location of both biosensor components. A set of test inhibitory RNA aptamers varying in length between 10 and 100 nucleotides are chemically synthesized and can be modified to contain either or both a cellular membrane transport peptide (e.g., antennapedia protein fragment) and a fluorescent label (e.g., fluorescein, rhodamine, GFP, etc.). Cells expressing the PPIB components are then treated with at least one of the inhibitory aptamers for a period of time ranging from 1 min to 24 h. Methods for treating cells with aptamers that do not contain cellular transport peptides can be loaded into cells using known membrane-perturbing approaches such as transient detergent solubilization, electroporation, microinjection, scrape loading, optical injection, etc. Furthermore, protein or RNA-based aptamers can be introduced into cells using expression vectors that can either be transfected or transduced with viral methods into living cells. Immediately after treatment with the test aptamers, the intracellular distribution of both the test aptamer and the shuttling p35 PPIB component is measured over time either kinetically or using a fixed end point approach. If the aptamer inhibits the interaction of the PPIB components, then the shuttling p35 biosensor component will distribute predominately to the cytoplasm and the inhibitory aptamer will distribute predominately with the PPIB component to which it is most strongly bound.

### Example 3

A Positional Biosensor for the Interaction of Full Length CdkS and p35.

In this embodiment, described is a protein-protein interaction biosensor (PPIB) to detect and measure the activity of compounds that disrupt the interaction of p35 protein with Cdk5, a tau activating kinase. The regulation of Cdk5 activity is pivotal not only to the phosphorylation of tau to induce its subsequent aggregation, but to the regulation of many other cellular processes, some of which play important roles in other neurodegenerative diseases. The kinase activity of Cdk5 is induced when it binds to the p35 protein. In some diseased cells, Cdk5 binds to a proteolytic degradation product of p35, the p25 protein. When bound to p25, Cdk5 kinase activity is improperly regulated and pathological phosphorylation levels of proteins such as tau occurs. Therefore, biosensors of the interaction between Cdk5 and p35 or p25 would be valuable reagents for use in screening protein complex disrupting compounds, especially those that may exhibit differential activity with p35 and p25.

Thus, it would be advantageous to produce protein-protein interaction biosensors (PPIBs) that measure the activation of Cdk5 by its necessary auxiliary protein p35 and its pathological degradation product p25. These biosensors provide a key drug target for a potentially large number of diseases. Cdk5:p35 and Cdk5:p25 PPIBs will also become foundation reagents for use in multiple cellular systems biology models of neurodegenerative disease.

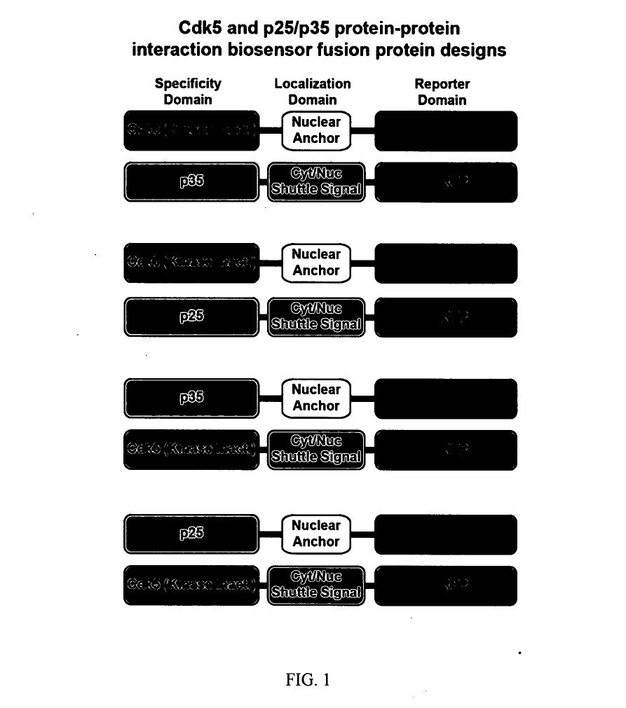

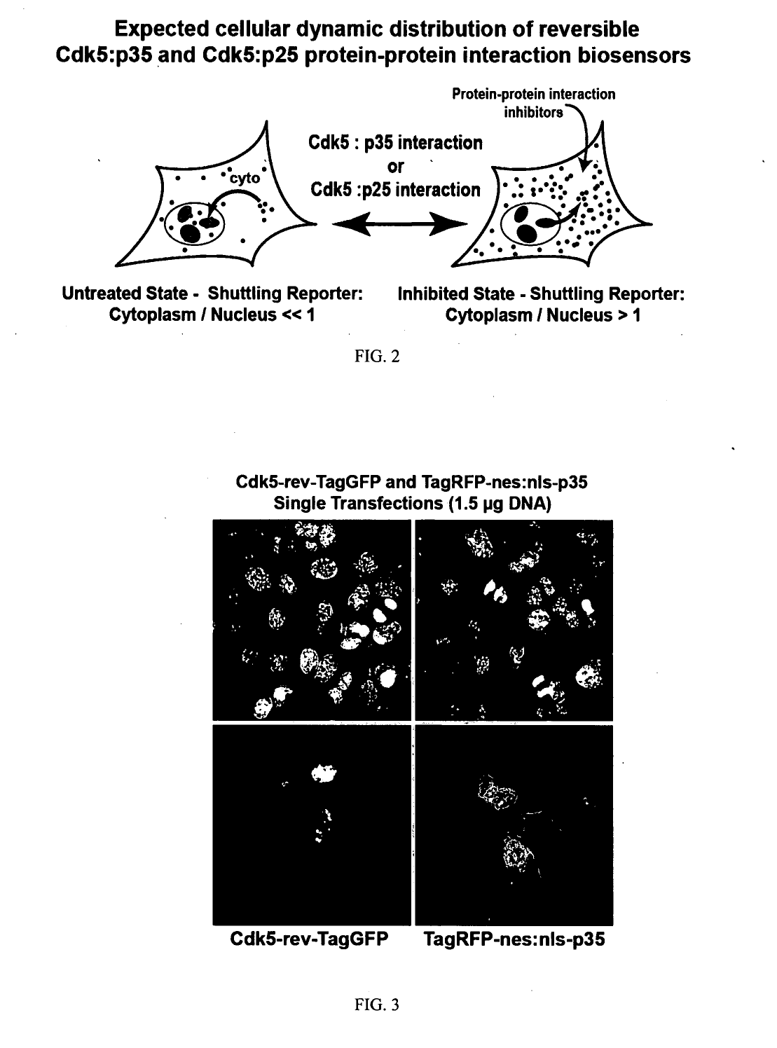

In one embodiment some of the designs of Cdk5:p35 are shown schematically in FIG. 1. These two-color, two-component biosensors are expressed in cells and are designed to report on protein-protein interactions through alterations in their intracellular location. To date, more than 20 vectors encoding full length Cdk5 (both kinase active and kinase inactive), p35, and p25 have been constructed. In some embodiments, vectors were prepared that would allow for either the Cdk5 or the p25-p35 proteins to be either predominately nuclear localized or nuclear-cytoplasmic shuttling. Furthermore, some vectors were built to also encode either a red or green fluorescent protein as a reporter of the location of each biosensor component within cells. FIG. 2 shows a model of the Cdk5:p35 PPIB mechanism of action. Treatment of cells with inhibitors of a specific protein-protein interaction induces a re-partitioning of one of the biosensor components from the nucleoli to the cytoplasm, an intracellular translocation that is easily quantified on a large scale with high throughput using high content screening technology.

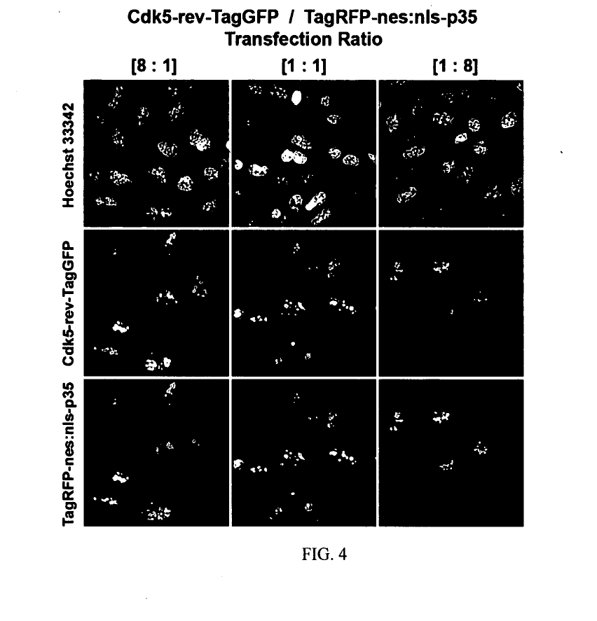

To first characterize the PPIB, cells were transfected with vectors encoding only one each of the biosensor components. FIG. 3 shows that in untreated cells, the biosensor components, when expressed alone, exhibited the expected localization in the cells. FIG. 4 demonstrates the interaction of an example pair of biosensor components when they were co-expressed. The biased partitioning of both biosensor components into the nuclear compartment over a wide range of biosensor component expression level is consistent with a strong interaction between the biosensor components. Thus, a disruptor of the Cdk5:p35 interaction will induce the measurable change in the distribution of the shuttling p35 component.

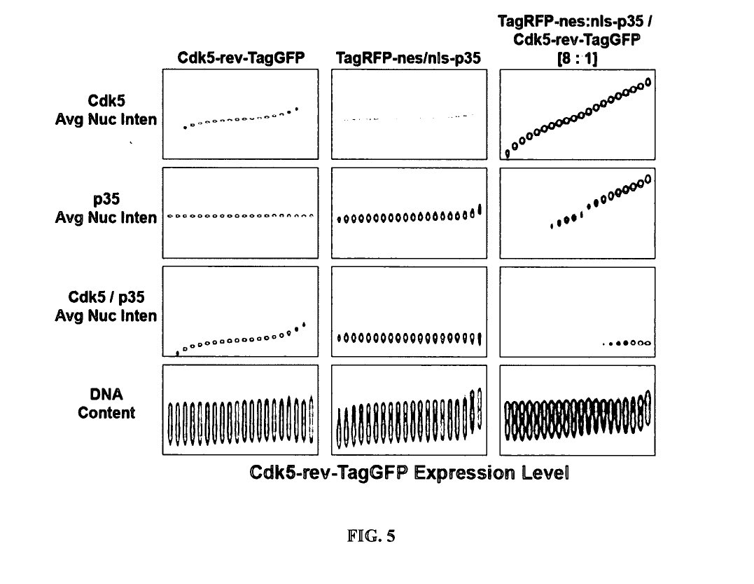

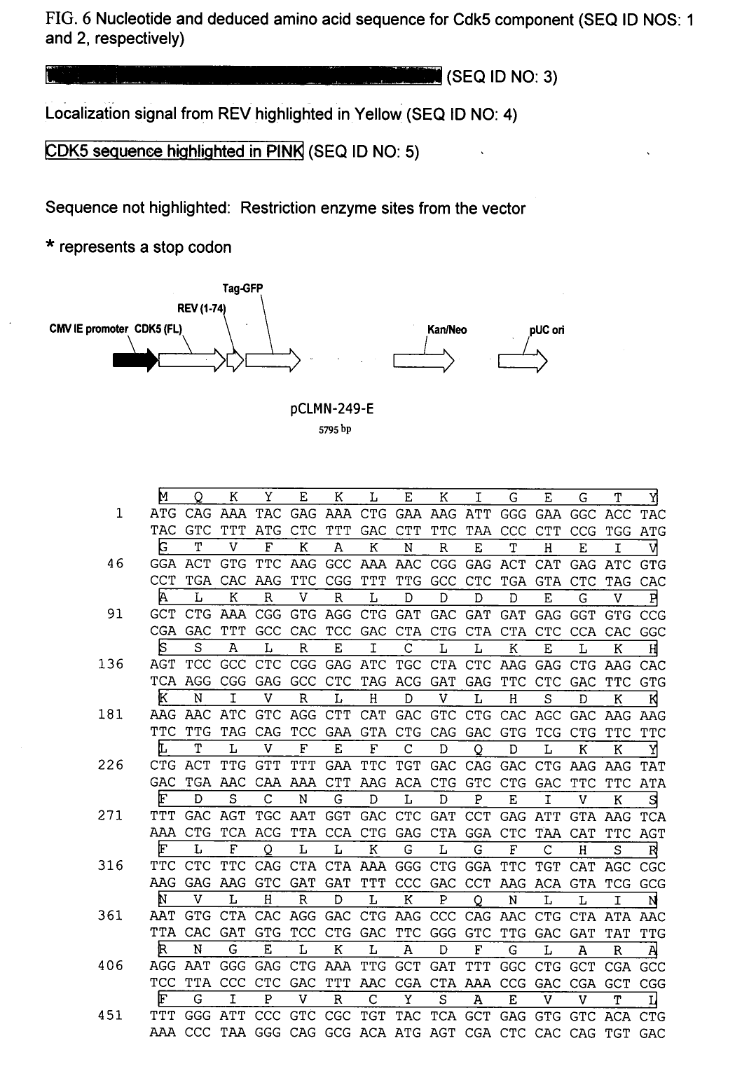

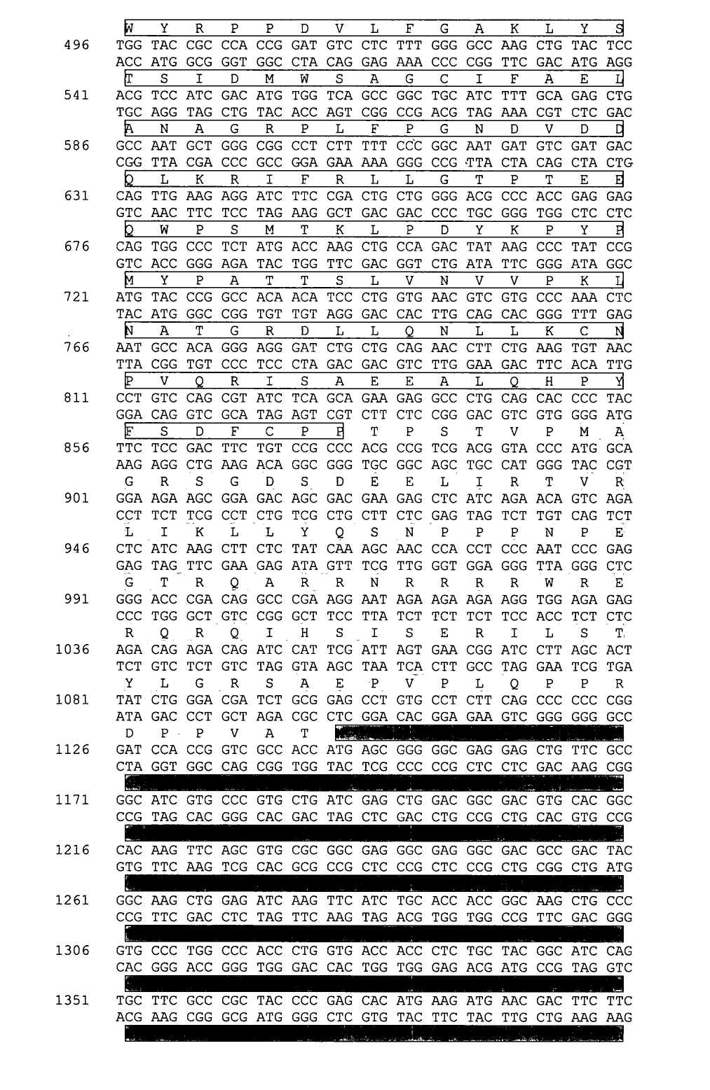

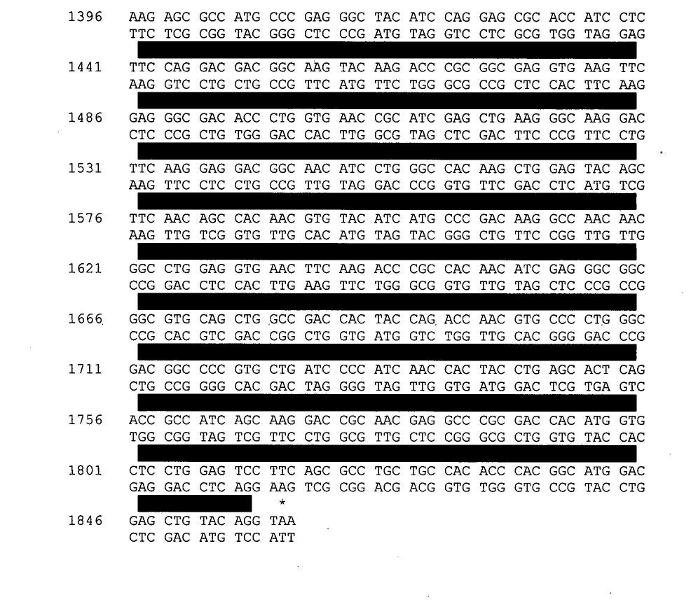

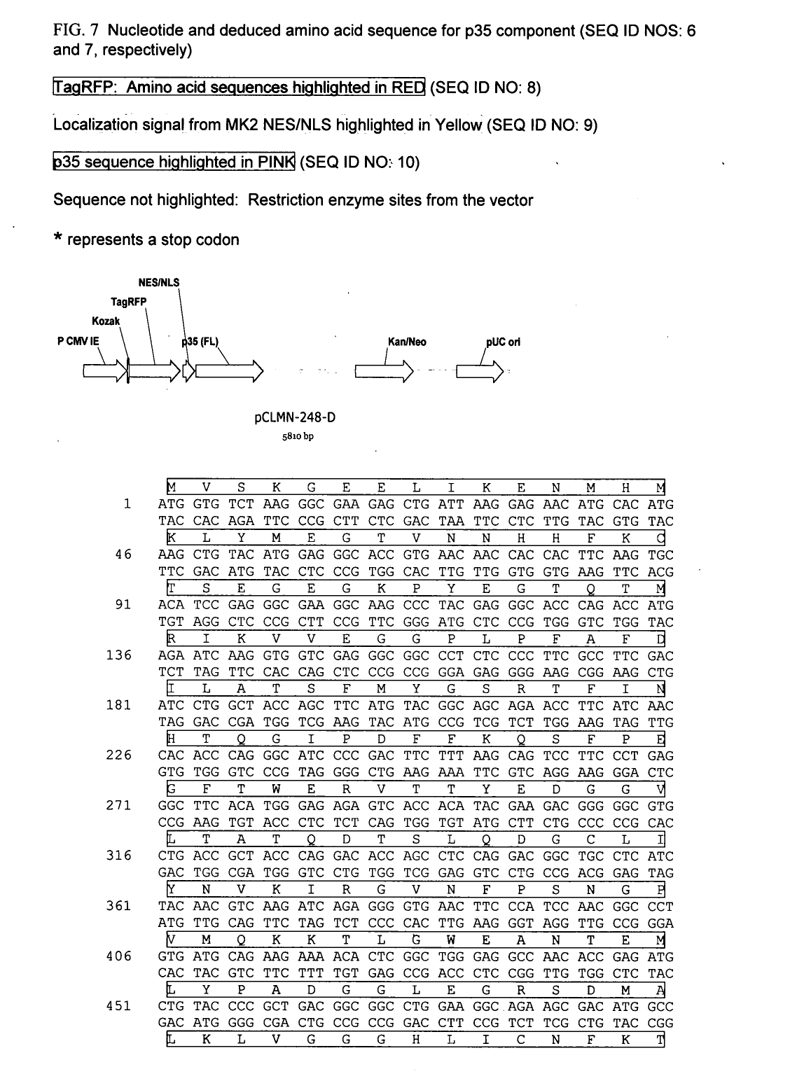

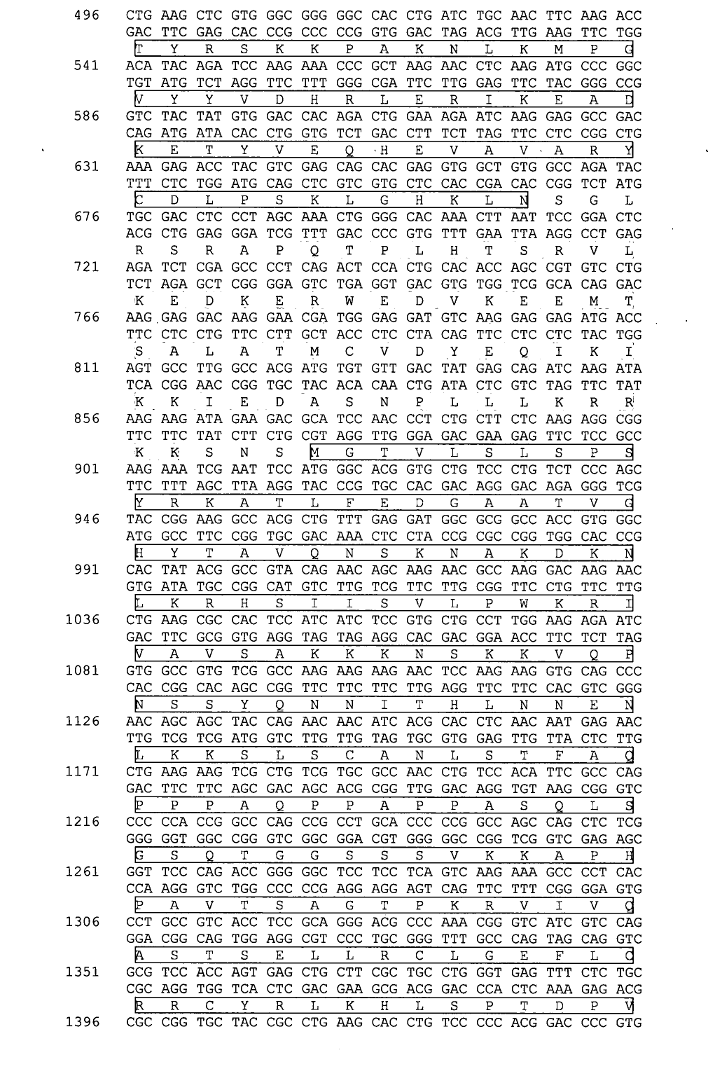

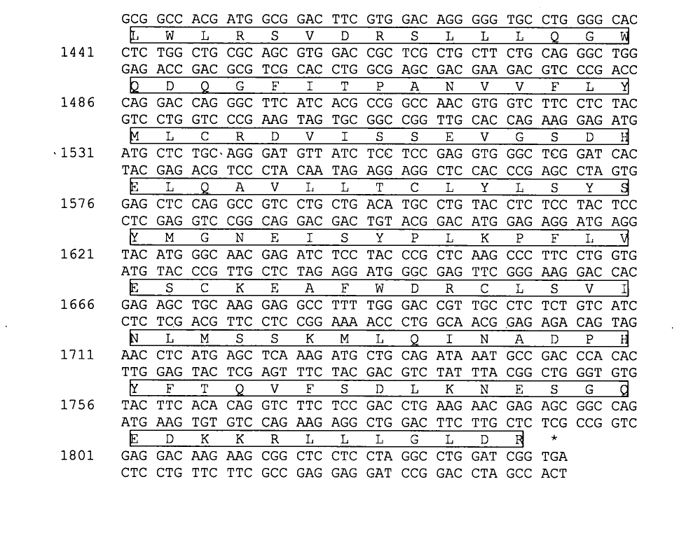

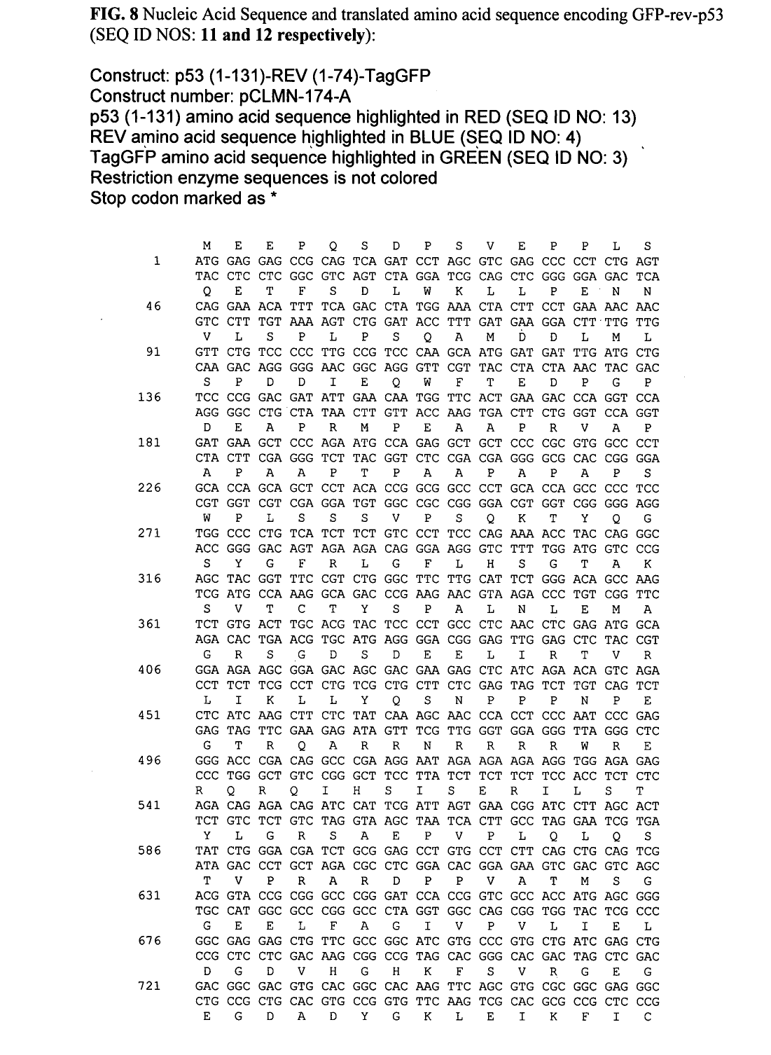

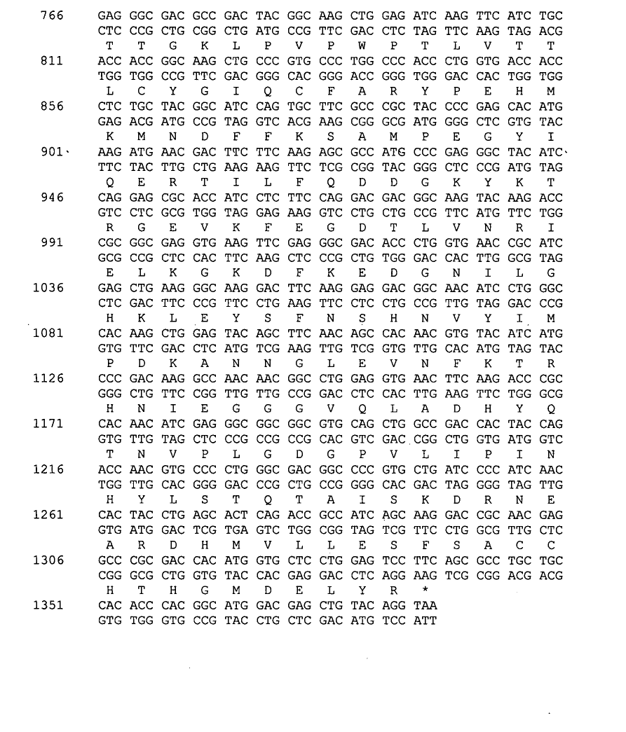

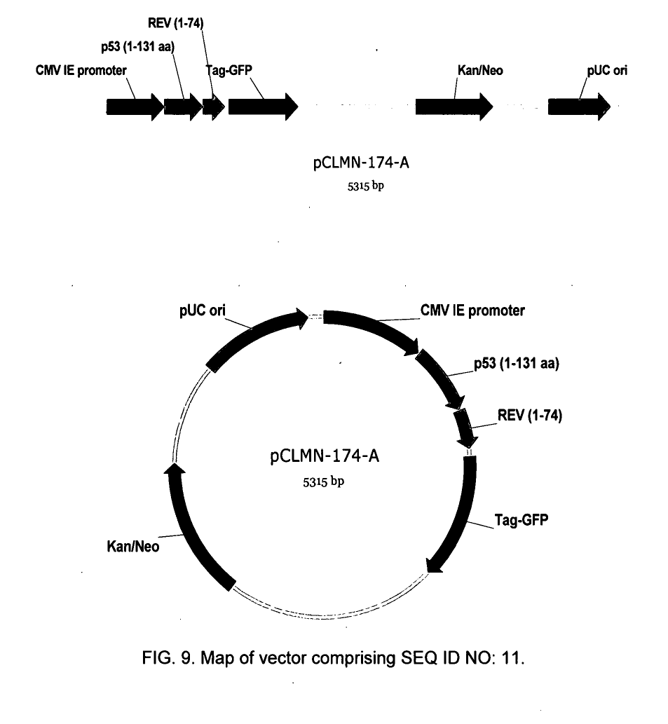

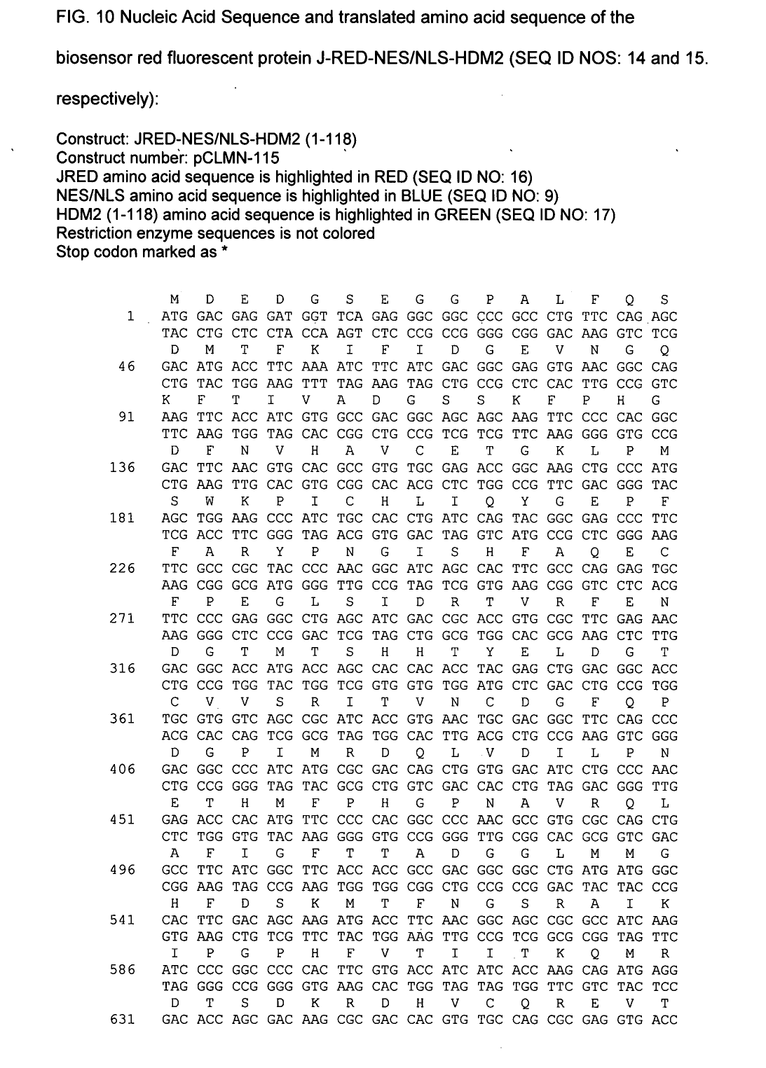

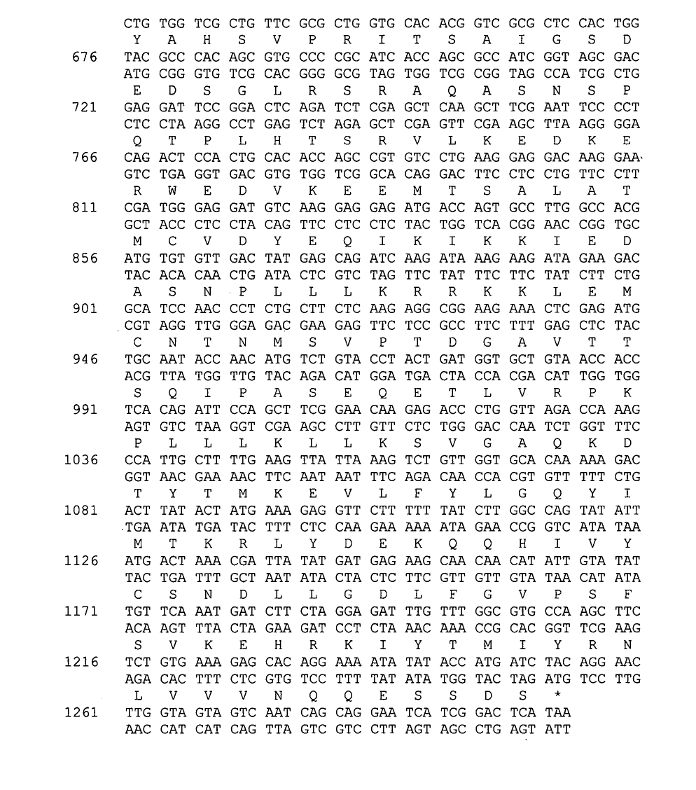

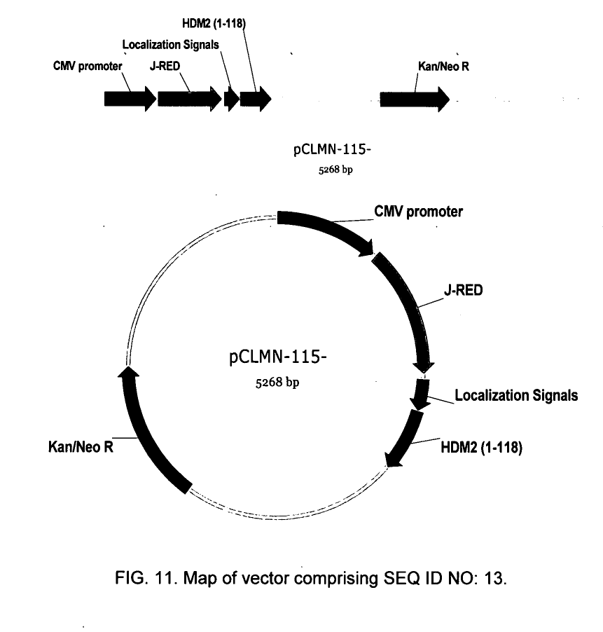

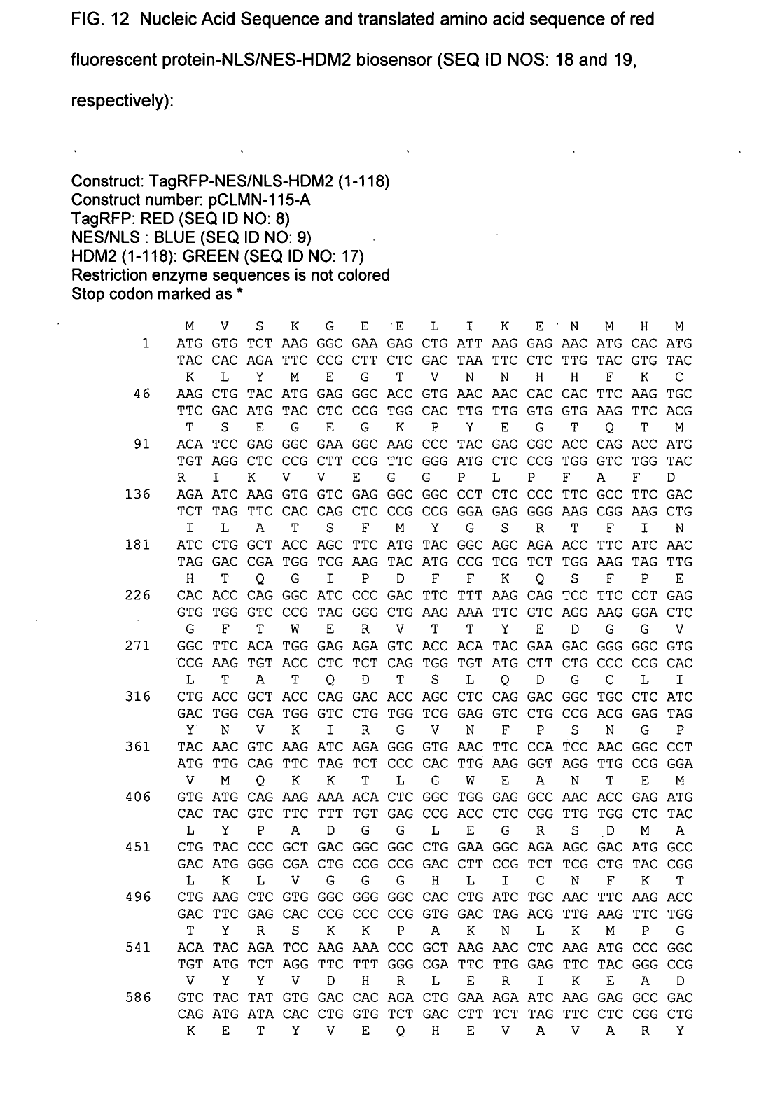

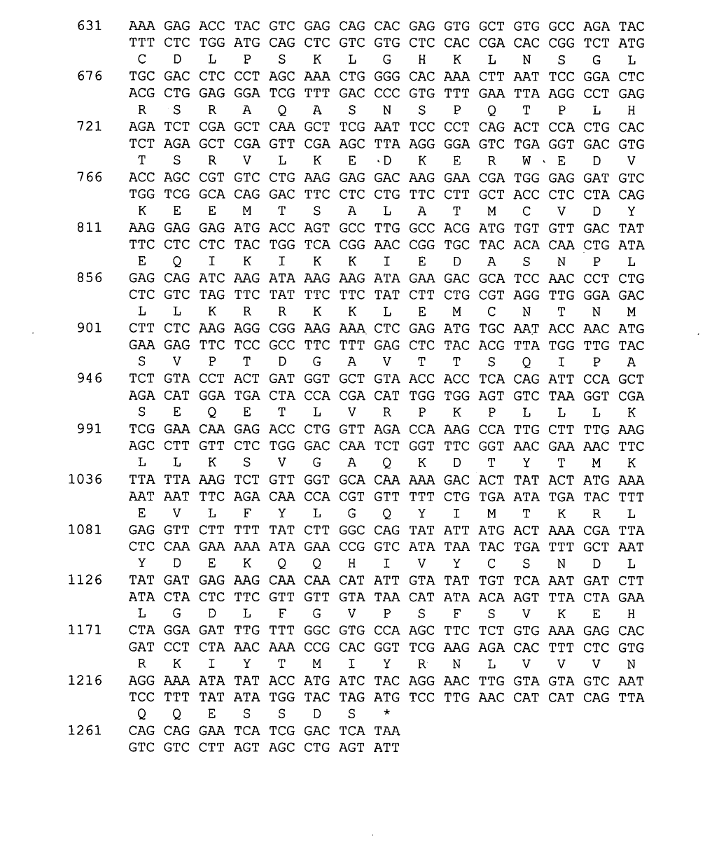

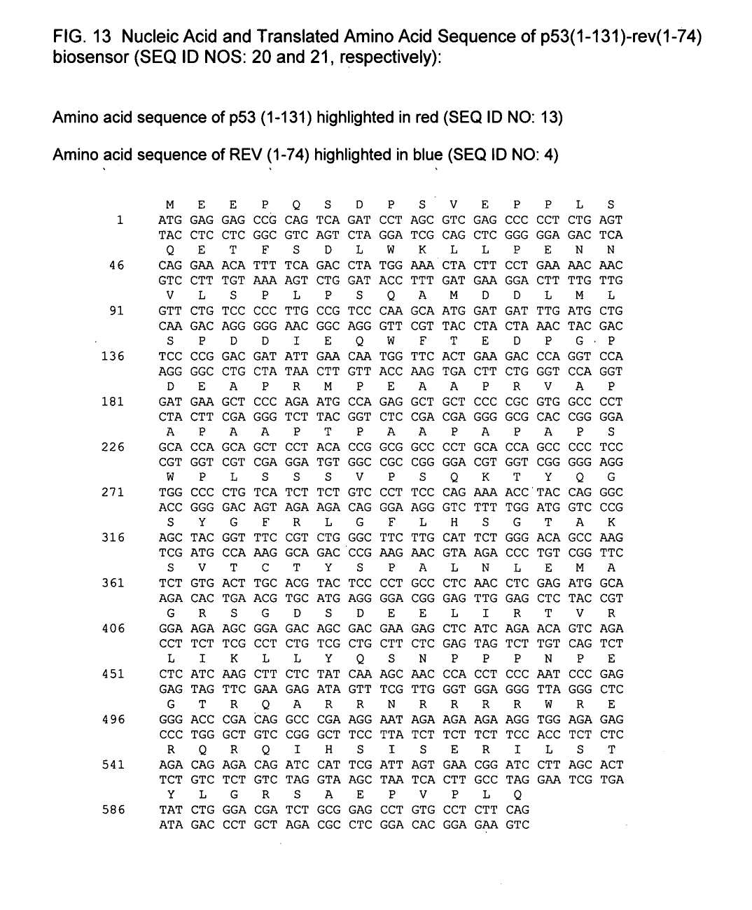

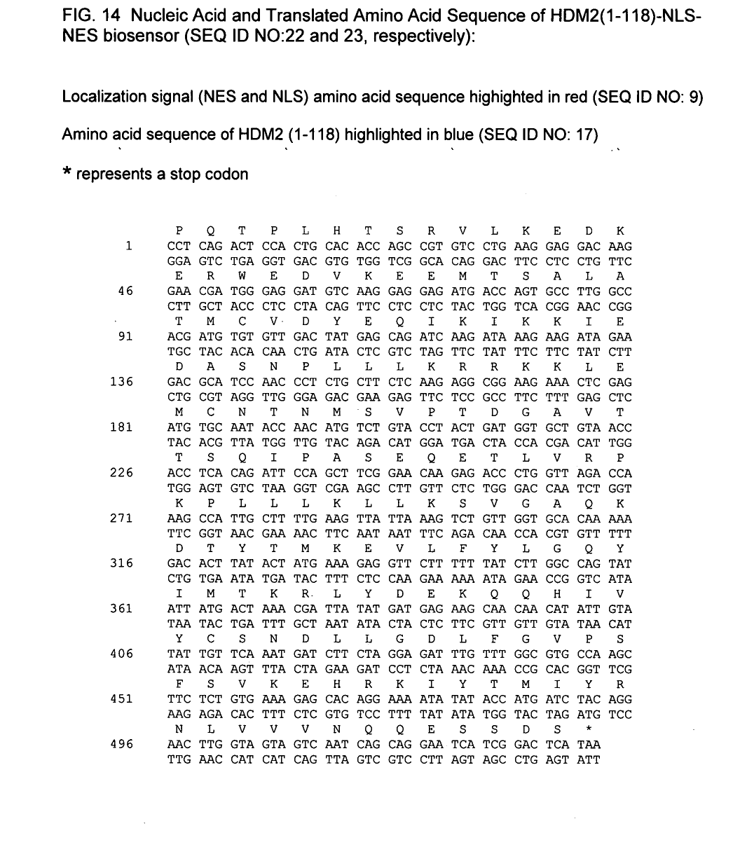

To further characterize the Cdk5:p35 PPIB, the expression level of both biosensor components, their relative distribution, and the DNA content of the cells co-expressing both biosensor components were measured. FIG. 5 shows cell population distribution maps that report cell population responses as a function of the expression level of the green Cdk5 biosensor component, which is anchored in the nucleus. FIG. 6 shows the nucleotide and amino acid sequence for a particular Cdk5-p35 PPIB.

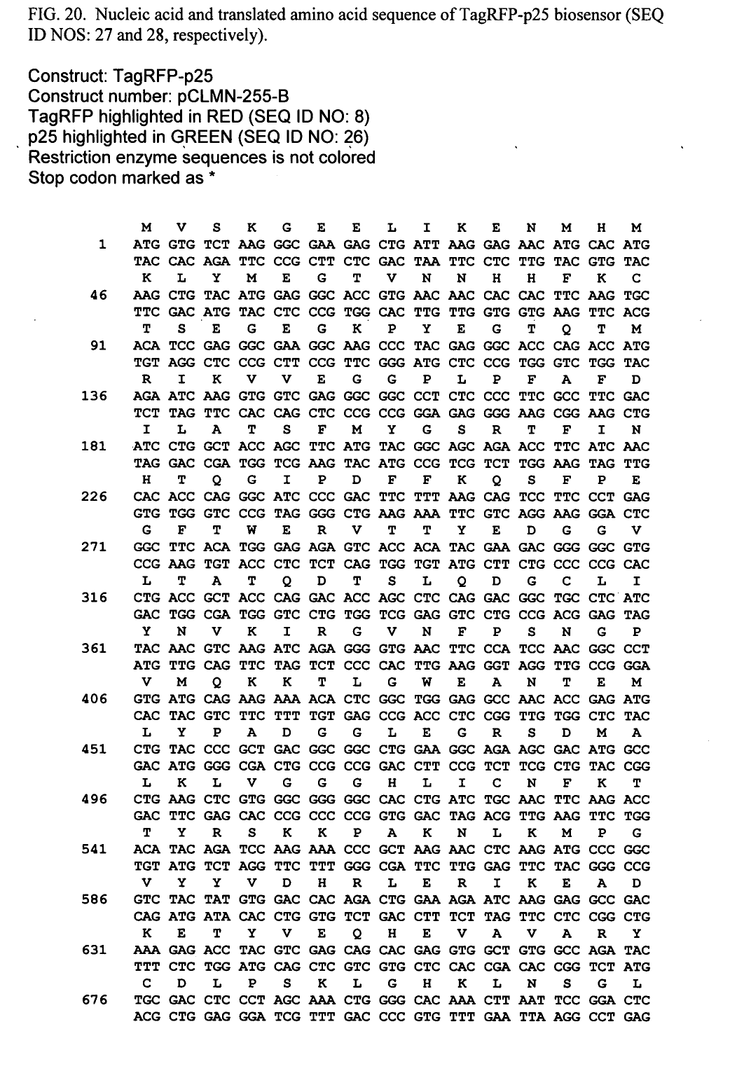

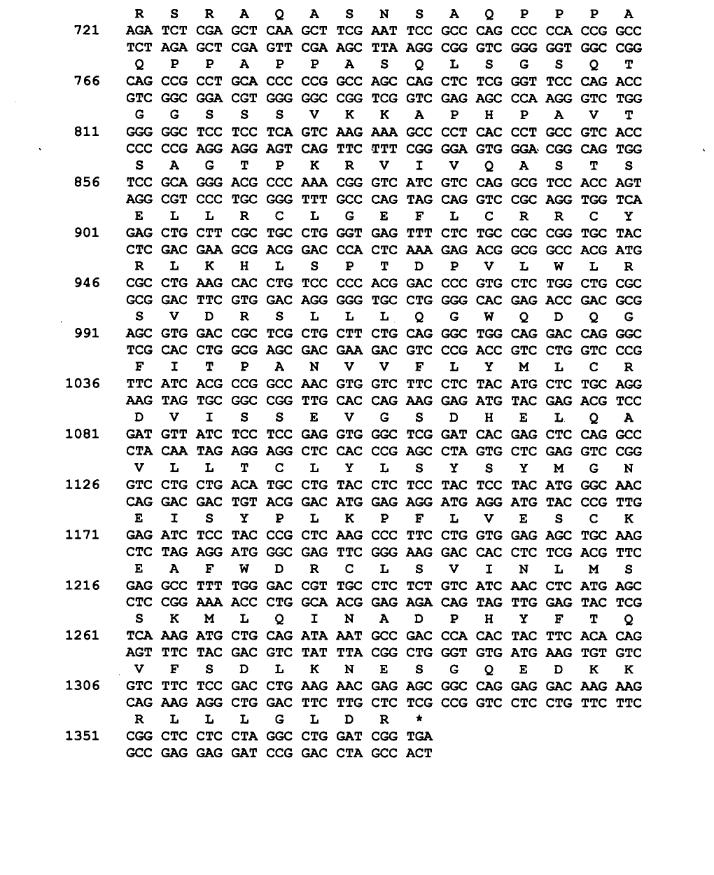

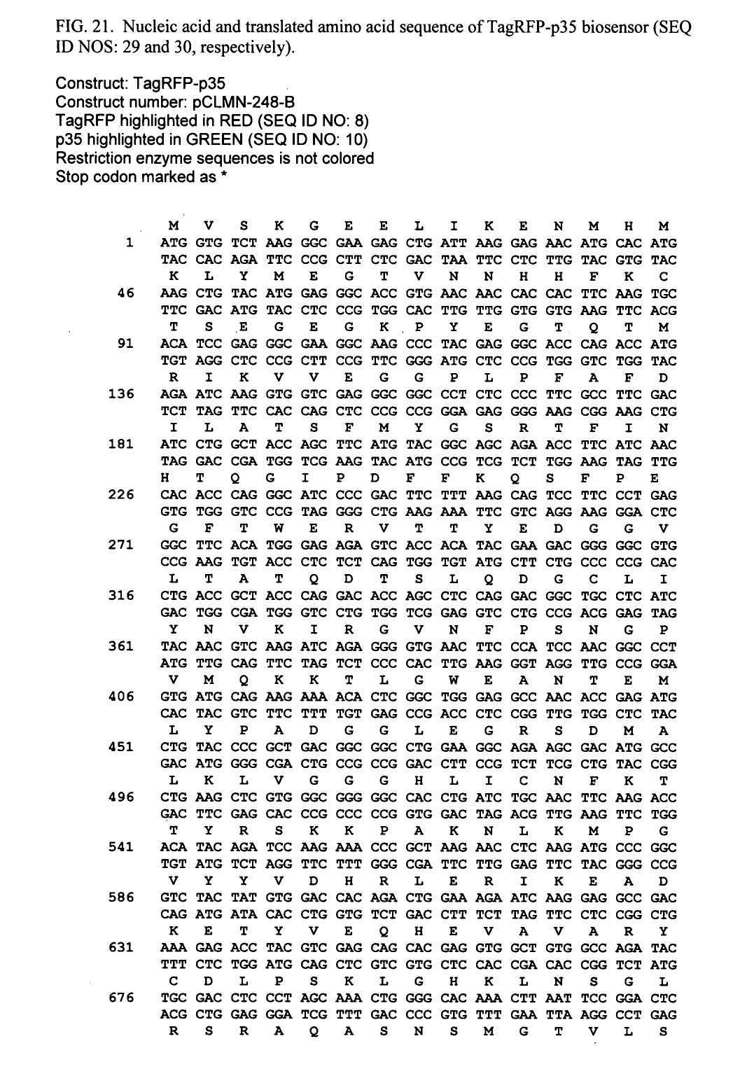

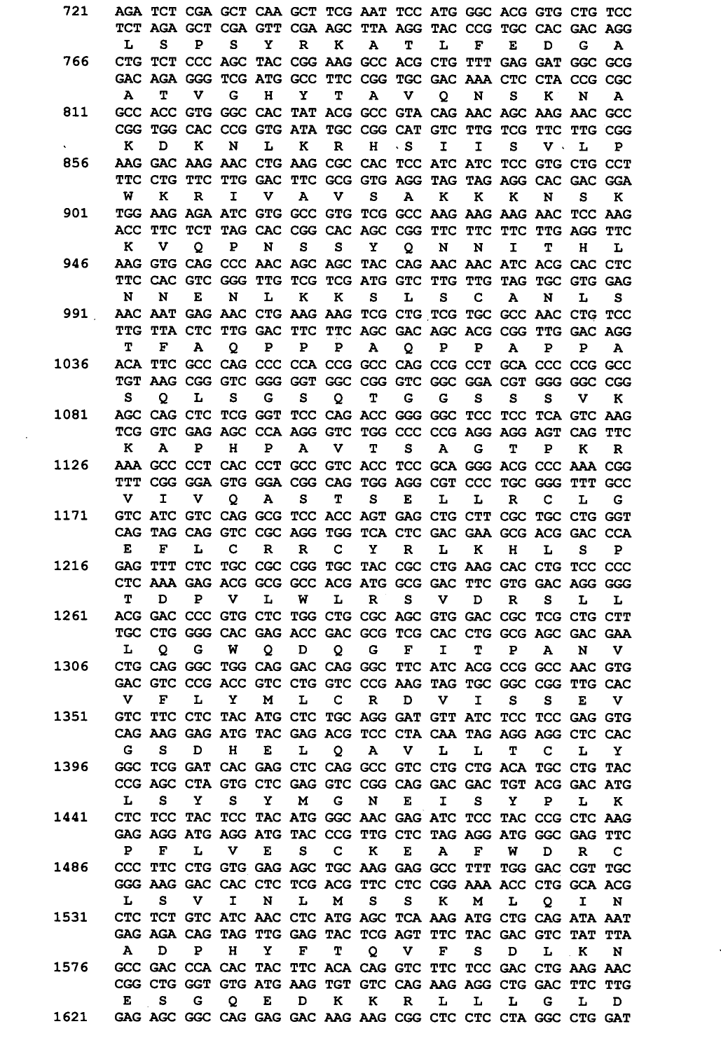

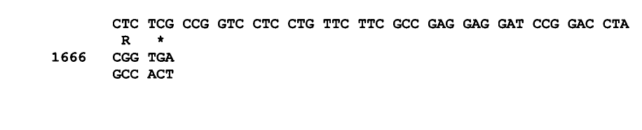

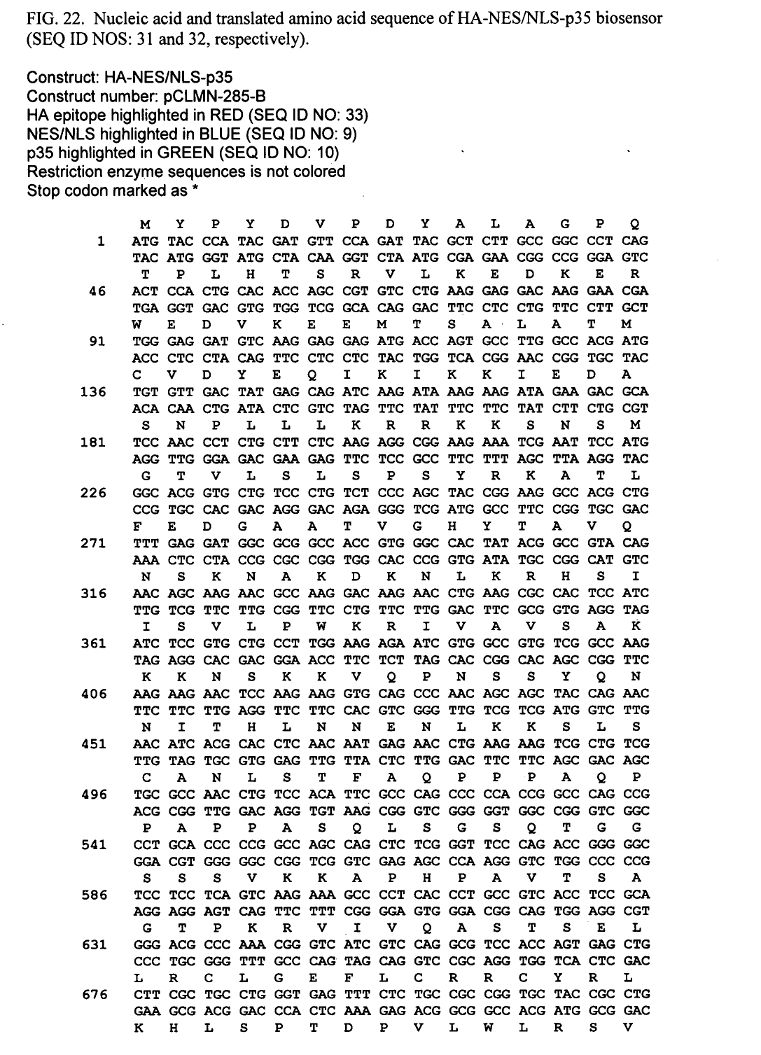

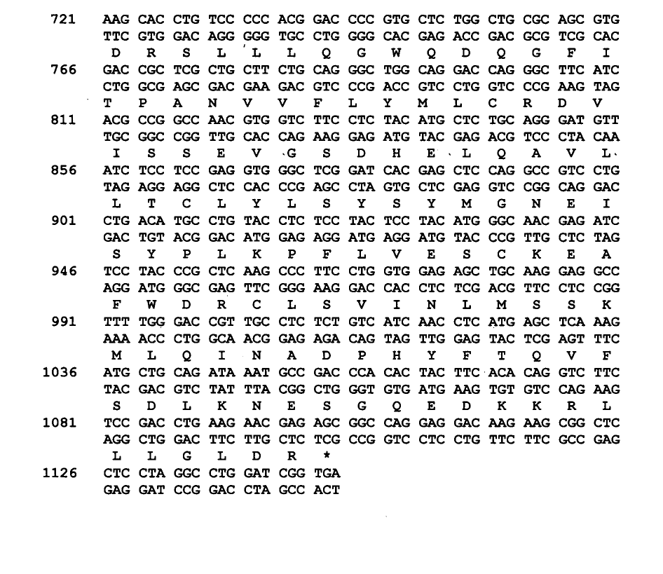

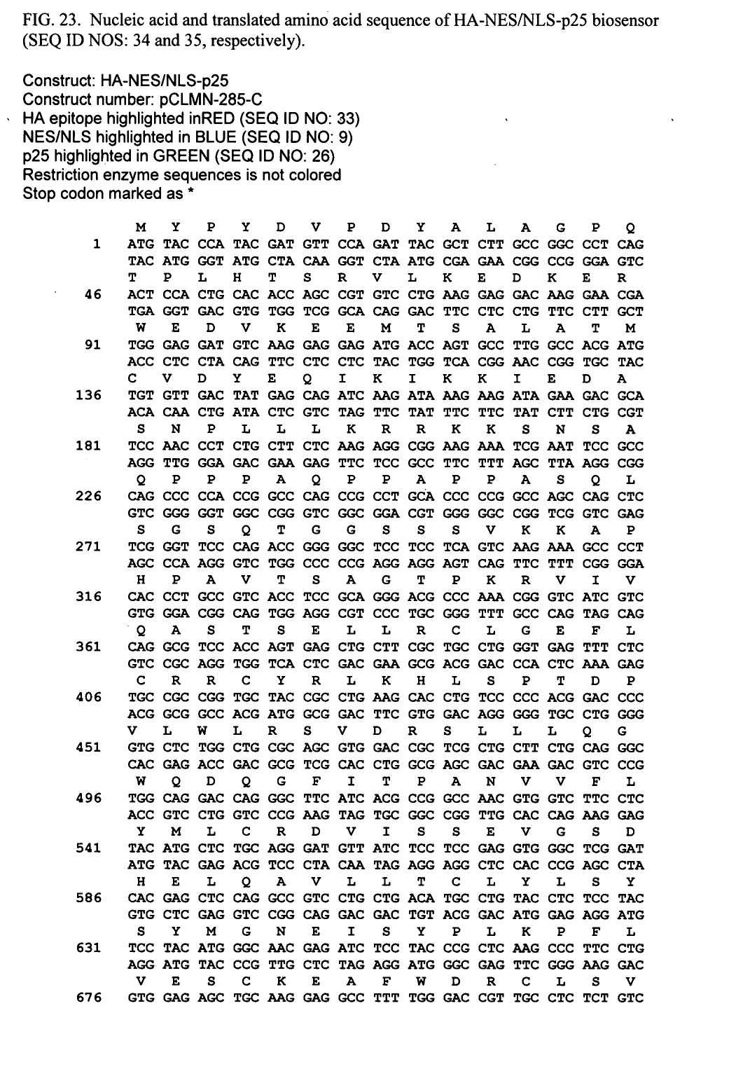

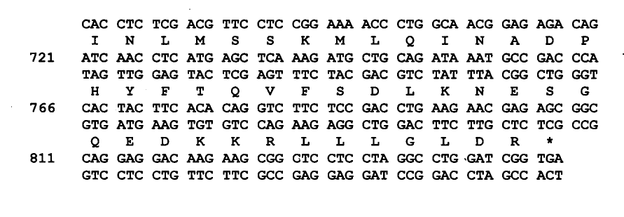

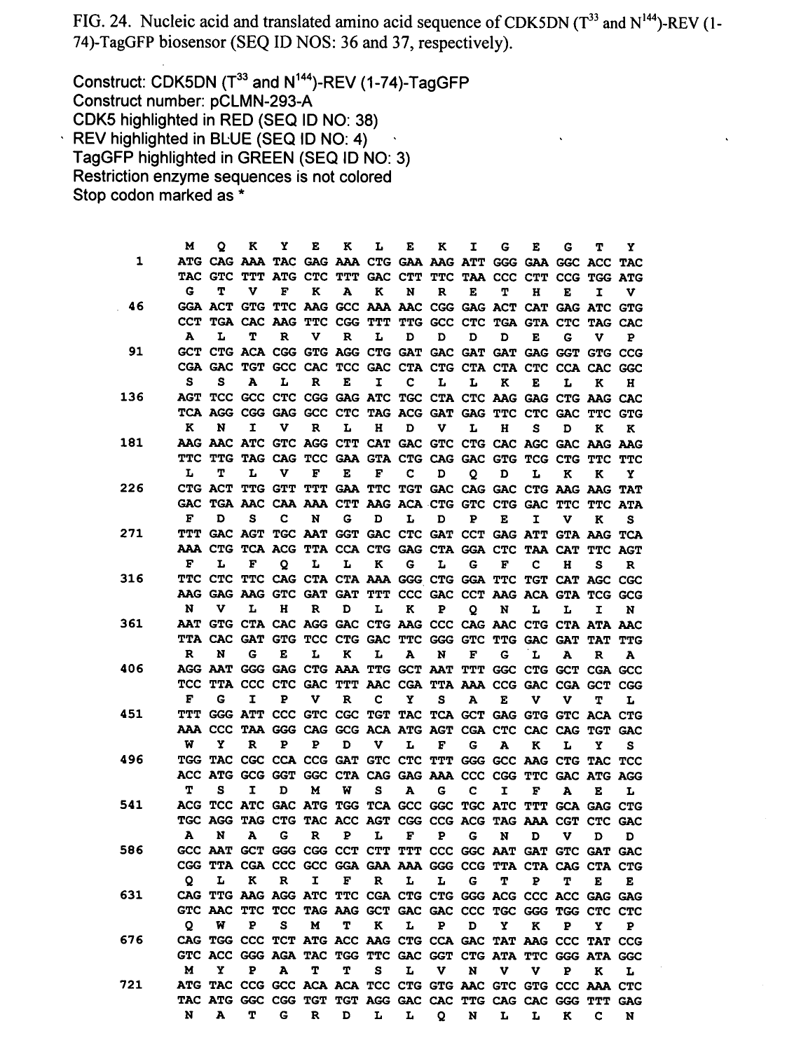

In another embodiment, cells were transfected with vectors encoding proteins similar to those shown in FIG. 3, but that contained only endogenous localization sequences (sequences illustrated in FIGS. 20 and 21). Endogenous localization sequences are those that are naturally found in the molecule of interest. As will be appreciated by the skilled artisan, many cellular molecules possess localization domains, such as nuclear localization domains, cytoplasmic localization domains, nucleolar localization domains, membrane localization domains, organelle localization domains, and the like. In one embodiment, the localization domain is endogenously encoded within the polypeptide comprising a binding domain and can comprise a nuclear localization domain, a nucleolar localization domain, a cytoplasmic localization domain, an organellar localization domain (such as a mitochondrial, peroxisomal and/or centrosomal), and a combination thereof. The binding domain of a molecule, such as a cellular polypeptide, can be associated with its natural localization domain as found in nature, without the necessary addition of an exogenous localization domain. When expressed alone, each protein exhibited the expected localization in the cells. Both the p25 and p35 biosensor components were distributed mostly cytoplasmically with a fraction distributed in the nucleus, but not nucleolus. When co-expressed with the CDK5 biosensor component, both the p25 and p35 biosensor components showed biased partitioning into the nuclear compartment consistent with a strong interaction between the biosensor components. Thus, a disruptor of the Cdk5:p35 or the CDK5:p25 interaction is predicted to induce the measurable change in the distribution of the p35 or p25 component.

### Example 4

Use of Positional Biosensors to Determine the Binding Domains that Regulate the Interaction of Cdk5 and p35.

In one embodiment, a first vector encoding full length Cdk5 fused to a localization domain and a detection domain is cotransfected into a cell with a series of second vectors encoding peptide sequences contained in the p35 protein ranging from about 2 amino acids up to and including full length p35 protein which are fused to a localization domain distinct from those encoded by the first vector. In another embodiment, the localization domain encoded by the first vector is from the rev protein which induces the protein to be predominately localized in the nucleus. Furthermore, the detection domain of the first vector encodes a fluorescent protein such as a green or red fluorescent protein. In one embodiment, a set of second vectors contain a localization domain encoded by the MAPKAP protein, which contains a pair of amino acid sequences encoding both a nuclear export and nuclear import signals such that the protein encoded by the second vector shuttles between the nucleus and cytoplasm with a predominate cytoplasmic location. Furthermore, the detection domain of the second vector encodes a fluorescent protein distinct from the detection domain encoded by the first vector.

The first vector is mixed with one of the second vectors and the pair is co-transfected into the same population of cells. In another embodiment, the first and second vectors are delivered into cells using a virus-based expression system. The location of the protein coded by the first vector is compared to the location of the protein encoded by the second vector using any suitable method available in the art, e.g., microscopic imaging methods. For example, the ArrayScan HCS reader produced by Thermo-Fisher ca be used to quantify the relative intracellular location of the two proteins. Co-localization of the two biosensor polypeptides in the same cellular compartment is consistent with there being an interaction between the two proteins that is stable enough to occur under normal intracellular conditions. In one example, examination of the p35 protein sequences encoded by the second vector that result in co-localization with the Cdk5 protein provides a list of p35 amino acid sequences that interact directly with full length Cdk5. In another embodiment, a first vector encoding full length p35 is tested with a second set of vectors encoding various fragments and full length sequences from Cdk5 to provide a list of Cdk5 amino acid sequences that interact directly with full length p35. In yet another embodiment, vectors encoding partial amino acid sequences of both Cdk5 and p35 are tested to determine which domains of each protein form stable complexes under normal intracellular conditions.

In yet another embodiment, compounds can be added to cells expressing the interacting Cdk5 and p35 domains and changes in the location of biosensor components can be used to measure the effect of the compounds on the interaction between Cdk5 and p35 domains.

### Example 5

A Three Component PPIB to Measure the Interaction of the Cdk5-p35 Complex with Tau Protein in Living Cells.

The regulation of the phosphorylation activity of the cyclin dependent kinase Cdk5 depends on its binding to the p35 protein, or the p25 protein, a proteolytic degradation product of p35. The active Cdk5-p35 (Cdk5-p25) complex has the ability to phosphorylate many substrates, of which tau protein is one. Tau protein, a microtubule associated protein, has been implicated to play a role in at least one disease, Alzheimer's disease. However, the art lacks the reagents and methodology to measure the dynamic interaction between the three proteins tau, Cdk5, and p35 (p25) in living cells. A PPIB to measure the interaction of the Cdk5/p35 (Cdk5/p25) complex with tau protein in cells would provide a valuable platform for understanding the regulation of the three-component protein complex as well as the effects that potential therapeutic compounds have on the stability of the three-component protein complex.

In one embodiment, a first expression vector is constructed that encodes full length, or suitable fragment thereof, Cdk5 as the binding domain fused to a localization domain, e.g., a nuclear localization domain and reporter domain, e.g., a green fluorescent protein (GFP) reporter domain (Cdk5-GFP). A second expression vector encoding a full length, or suitable fragment thereof, p35 protein as the binding domain fused to a reporter domain, e.g., a red fluorescent protein (RFP) reporter domain (p35-RFP) is also constructed. Finally, a third expression vector is constructed encoding full length, or suitable fragment thereof, tau as the binding domain fused to a localization domain, e.g., a nuclear-cytoplasmic shuttling (NES/NLS) sequence, and reporter domain, e.g., an epitope tag (HA; hemaglutin) (tau-HA). In this embodiment, all three expression vectors are introduced into the same population of cells. When co-expressed, the p35-RFP will partition predominately into the nucleus because it will be bound to the nuclear-anchored Cdk5-GFP protein. Furthermore, the tau-HA will partition predominately into the nucleus because its interaction with the Cdk5-GFP:p35-RFP complex will dominate the NES/NLS shuttling sequence that normally induces net translocation of protein cargo to the cytoplasm. Upon disruption of the interaction between the Cdk5-GFP:p35-RFP complex and tau-HA, the tau-HA biosensor component will be free to exhibit a net translocation to the cytoplasm. A high-content screening reading of the nuclear-cytoplasmic distribution ratio of the tau-HA biosensor component will provide a measurement of the disruption of the Cdk5-GFP:p35-RFP complex interaction with tau-HA. The ratio will decrease upon disruption of the ternary protein complex.

### Example 6

Many procedures discussed herein, such as luminescence and/or fluorescence tagging and detection, PCR, vector construction, including direct cloning techniques (including DNA extraction, isolation, restriction digestion, ligation, etc.), cell culture, transfection of cells, protein expression and purification, and HCS assays are techniques routinely performed by one of ordinary skill in the art (see generally Sambrook et al., Molecular Cloning, A Laboratory Manual, Cold Spring Harbor Laboratory, Cold Spring Harbor, NY 1989).

This example demonstrates the construction and optimization of a modular biosensor to measure a specific protein-protein interaction in living cells. This biosensor is constructed to analyze the dynamic complex formation between the p53 tumor suppressor protein and its major intracellular binding partner, the HDM2 protein, which is the human homolog of mouse MDM2. The approach outlined here can, however, be applied to the construction of other biosensors.

A eukaryotic expression plasmid that encodes a biosensor comprising SEQ ID NO: 11 having an appropriate nucleolar localization sequence, a fragment of the p53 protein, and a green fluorescent protein was constructed. A separate expression vector comprising SEQ ID NO: 18 encoded a red fluorescent protein joined with an appropriate nuclear export and nuclear import sequence combination was further joined with the coding sequence for a fragment HDM2. Co-transfection of the two plasmids into human tumor cells (U2OS) expressing wild type p53 produced cells with p53-HDM2 complexes distributed predominately in the nucleoli. Upon treatment with a disruptor of the p53-HDM2 interaction (e.g., nutlin-3), the NLS-p53-GFP construct redistributed predominately into the cytoplasm.

Preparation of cells expressing rev-p53-GFP and NES/NLS-HDM2-RFP: To produce cells expressing biosensors, a standard strategy for the transient double transfection of mammalian cells was used. Briefly, U2OS cells were grown at log phase and aa population (4×10⁺⁶) were transfected with a mixture of expression plasmids encoding SEQ ID NOS: 12 and 19 at a 4:1 mass ratio (2 μg total) using Amaxa nucleofection reagents and electroporation. After an 18-24 hour incubation, the transfected cells were trypsinized and plated at 6000-8000 cells per well in collagen 1 coated 384-well microplates (Falcon #3962). Cells at this stage were ready for use in either live cell kinetic or fixed end point HCS assays.

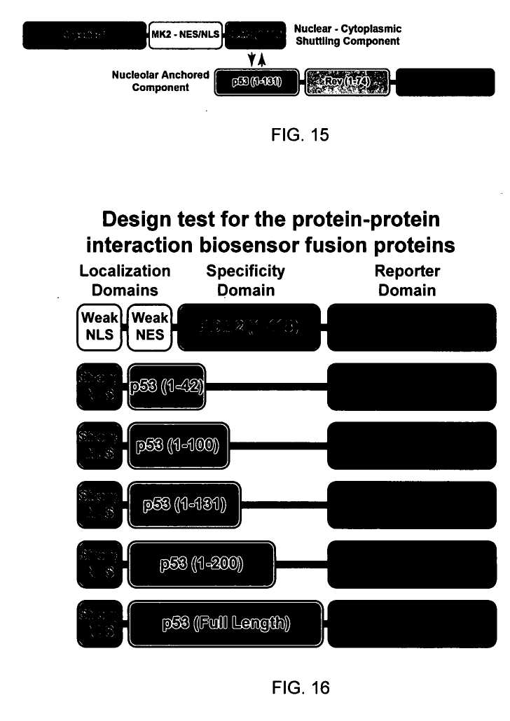

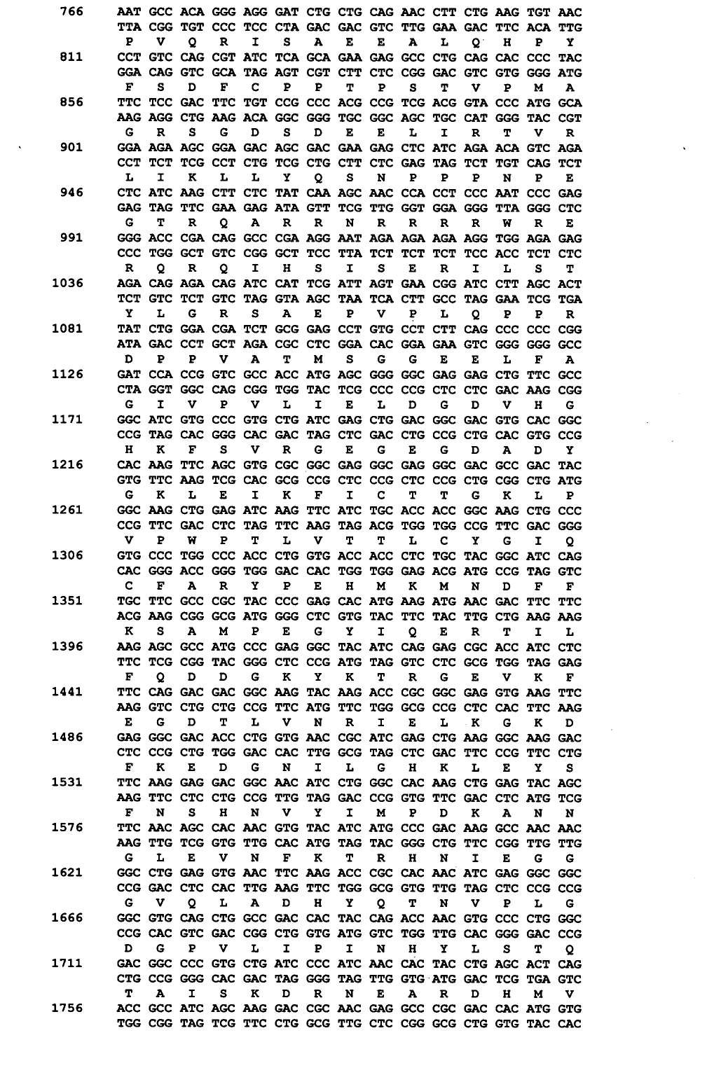

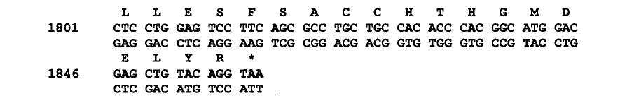

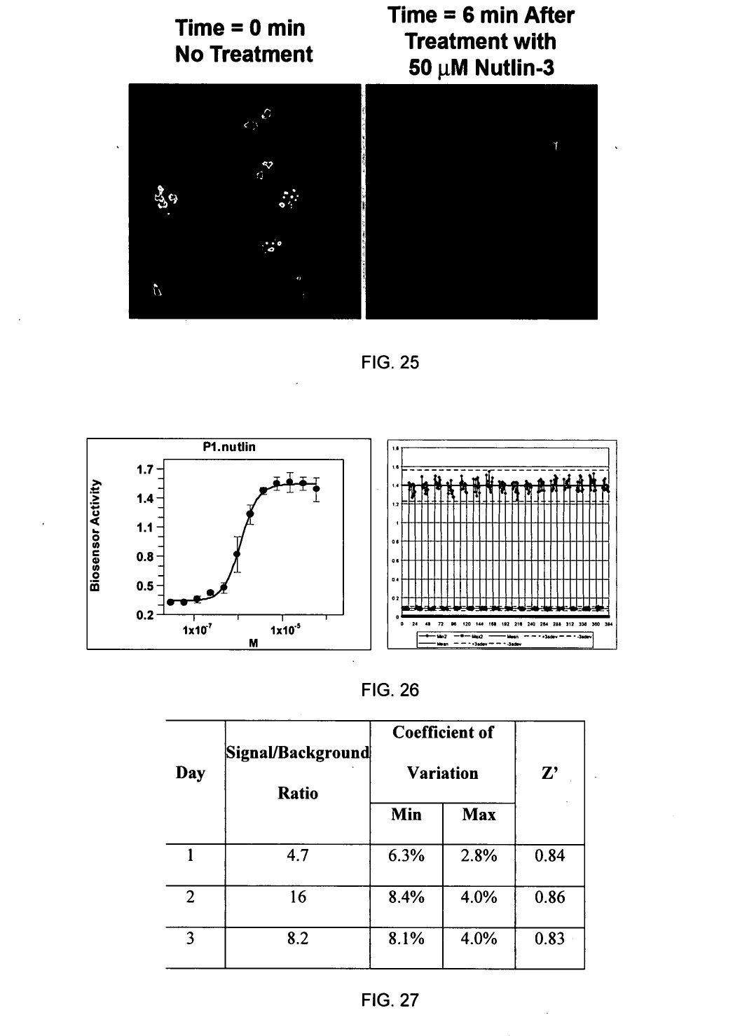

The p53:HDM2 protein-protein interaction biosensor (PPIBs) is shown schematically in FIG. 15. These two-color, two-component biosensors were expressed in cells and were designed to report on protein-protein interactions through alterations in their intracellular localization. FIG. 2 shows a model of PPIB mechanism of action. Treatment of cells with inhibitors of a specific protein-protein interaction induces a re-partitioning of one of the biosensor components from the nucleoli to the cytoplasm, an intracellular translocation that is easily quantified on a large scale with high throughput using high content screening technology. To demonstrate the utility of the PPIB, cells were transfected with vectors encoding the two-component PPIB. FIG. 25 (left panel) shows that in untreated cells, the shuttling component of the biosensor was localized in the nucleoli where it strongly interacted with the other biosensor component which was anchored in the nucleoli. Within minutes after treatment with nutlin-3, the nucleolar fluorescence signal dispersed and re-partitioned into the cytoplasm of the same cells (FIG. 25, right panel). Using washout experiments, the drug-induced translocation of the biosensor was reversible.

The p53:HDM2 PPIB was incorporated into an HCS assay and the assay validated to industry standards. FIG. 26 shows example data from the validation data set. The response of the biosensor to nutlin-3 activity was reproducible and exhibited an EC50 of 1.1 μM (FIG. 26, left panel). FIG. 26 also shows that an assay incorporating the PPIB showed acceptable intra-plate variability with a Z′ of 0.86. The three-day interpolate variability of the PPIB in an HCS assay was also acceptable according to industry standards. The Z′ values were consistently >0.8 (n.b., Z′ values >0.25 are considered acceptable) and the coefficient of variation values of all three days were well below the industry standard maximal values of 14%. Three day Intraplate variability data show that the assay incorporation the biosensor is robust (FIG. 27).

### Example 7

Using Intracellular Localization of Biosensor Components to Determine the Interacting Domains of p53 and HDM2

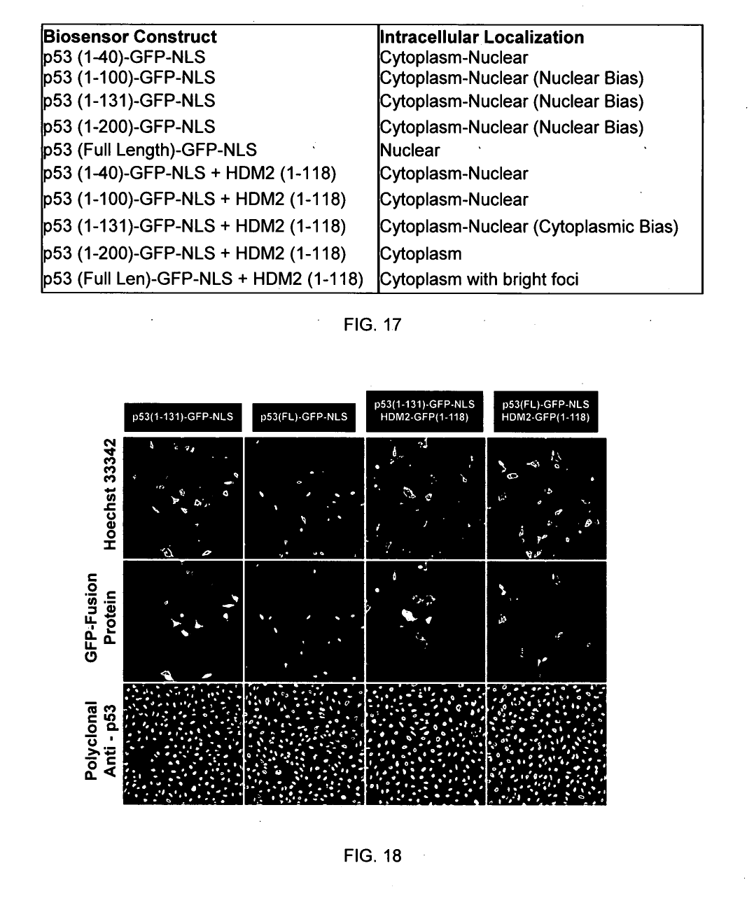

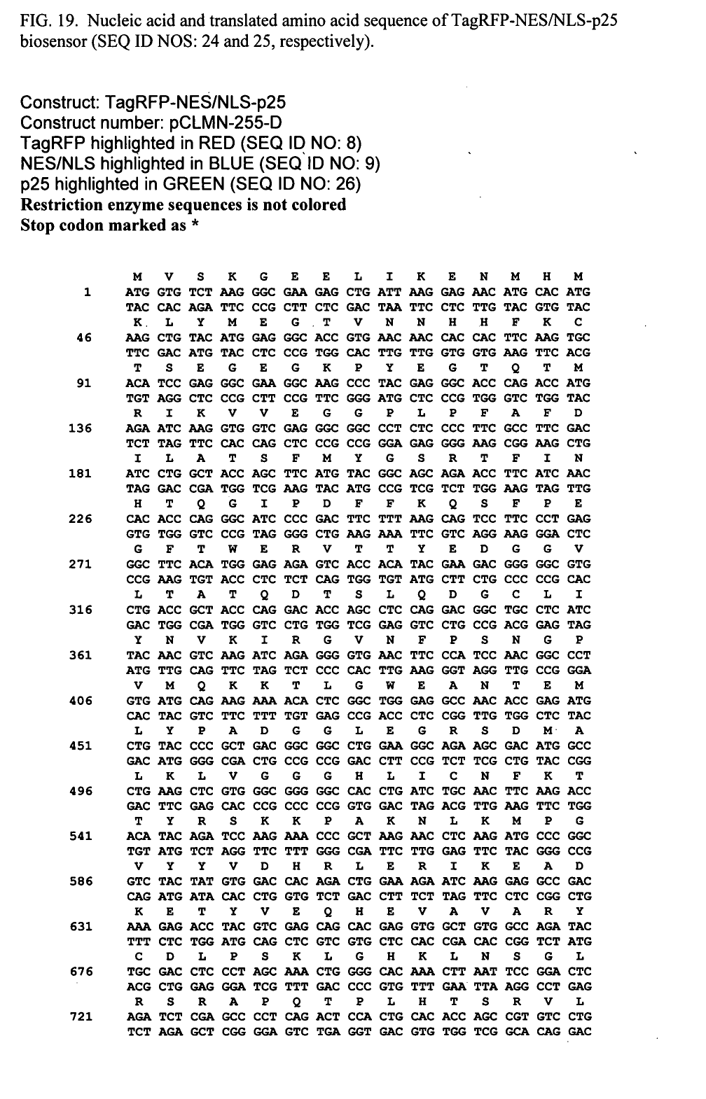

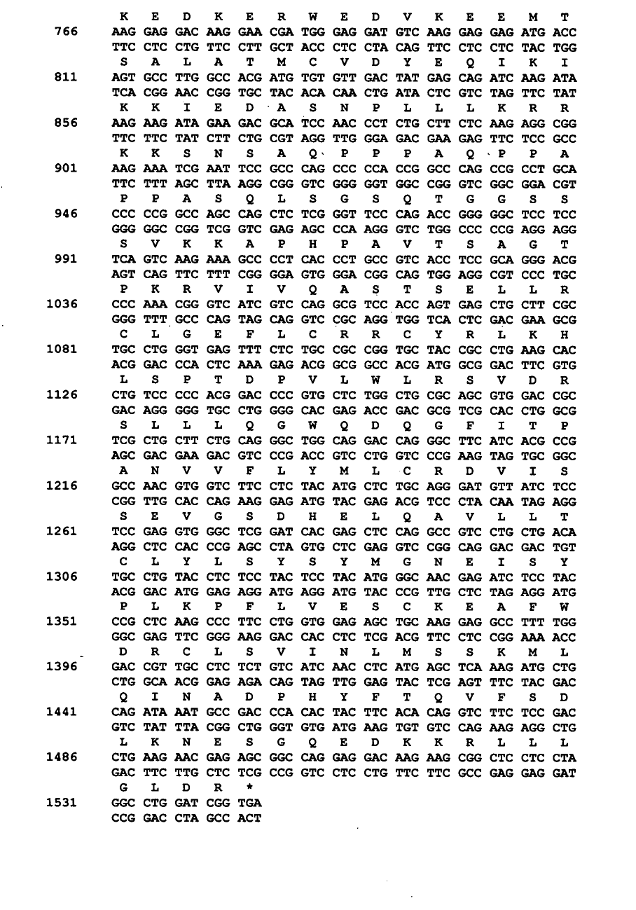

Five constructs were built that express several fragments of p53 as well as the full length protein, all fused with a strong NLS (SV40) and EGFP (FIG. 16). A construct encoding a cytoplasm-nuclear shuttling domain of HDM2 (1-118) was also built (FIG. 16). First, the p53-GFP-NLS constructs were expressed alone in U2OS cells and their distribution measured. The full length p53-GFP-NLS construct was the only biosensor component to be localized exclusively in the nucleus. The other constructs showed both cytoplasm and nuclear localization (FIG. 17). When co-expressed with the shuttling HDM2 construct, several of the p53-GFP-NLS proteins showed altered localization, consistent with interaction with the shuttling HDM2 protein. FIGS. 17 and 18 show that the longer the p53-GFP-NLS construct, the more likely it was to become localized in the cytoplasm, where the HDM2 protein fragment was predominately localized. Furthermore, the full length p53-GFP-NLS localized into cytoplasmic foci when coexpressed with the HDM2 protein fragment. Thus, assaying the intracellular localization of full length proteins and protein fragments within living cells provides information on their interaction in a natural environment. It also provides a framework to test treatments with the potential to modulate the interaction between the proteins and their fragments.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method for identifying an agent that modulates the interaction of two or more polypeptides, comprising:
a) introducing into a cell at least a first polypeptide and a second polypeptide, each comprising a binding domain, a localization domain, and a reporter domain, wherein the first polypeptide comprises a localization domain that is different from the localization domain of the second polypeptide;
b) maintaining the cell under conditions in which the binding domain of the first polypeptide interacts with the binding domain of the second polypeptide, which results in co-localization of the first polypeptide and the second polypeptide at a first cellular location in the cell;
c) introducing to the cell an agent; and
d) detecting the cellular location of the first polypeptide, the second polypeptide or a combination thereof, wherein a change in the cellular location of the first polypeptide, the second polypeptide or a combination thereof as compared to the cellular location in step (b) indicates that the agent modulates the interaction of the two or more polypeptides.

2. The method of claim 1, wherein the agent is a macromolecule, a small molecule, or a combination thereof.

3. The method of claim 2, wherein the macromolecule is a protein, peptide, nucleic acid, aptamer, simple carbohydrate, complex carbohydrate, fatty acid, lipid molecule, or a combination thereof.

4. The method of claim 1, wherein the agent is labeled with a cellular transport peptide, a fluorescent label, or a combination thereof.

5. The method of claim 1, wherein the localization domain of the first polypeptide and second polypeptide are independently selected from the group consisting of a nuclear localization domain, a nucleolar localization domain, a cytoplasmic localization domain, an organellar localization domain, and a combination thereof.

6. The method of claim 1, wherein the reporter domain of the first polypeptide and the reporter domain of the second polypeptide are the same or different and are selected from the group consisting of: a fluorescent protein and a tag.

7. The method of claim 6, wherein the tag is selected from the group consisting of a SNAP tag, a Halo tag, a Lumio, a FlAsH tag, and an epitope tag.

8. The method of claim 1, wherein the first polypeptide, the second polypeptide, and/or the agent are introduced into the cell by transfection, electroporation, optoinjection, membrane translocating signal sequence attachment, cell scraping, or detergent treatment of the cell.

9. The method of claim 1, wherein the first polypeptide comprises a binding domain of a first protein, and the second polypeptide comprises a binding domain of a second protein, wherein the first protein and second protein are different.

10. The method of claim 9, wherein the first protein is selected from the group consisting of a disease-associated protein, a non-disease associated protein, and a combination thereof.

11. The method of claim 11, wherein the disease-associated proteins are associated with cancer or neurodegenerative diseases.

12. The method of claim 9, wherein the second protein is selected from the group consisting of a disease-associated protein, a non-disease associated protein, and a combination thereof.

13. The method of claim 12, wherein the disease-associated proteins are associated with cancer or neurodegenerative diseases.

14. A method for identifying an agent that modulates the interaction of two or more polypeptides, comprising:
a) introducing into a cell at least a first polypeptide and a second polypeptide, each comprising a binding domain, a localization domain, and a reporter domain, wherein the first polypeptide comprises a nuclear localization domain and the second polypeptide comprises a nuclear-cytoplasmic shuttling localization domain;
b) maintaining the cell under conditions in which the binding domain of the first polypeptide interacts with the binding domain of the second polypeptide, which results in co-localization of the first polypeptide and the second polypeptide in the nucleus of the cell;
c) introducing to the cell an agent; and
d) detecting the cellular location of the second polypeptide, wherein a change in the cellular location of the second polypeptide from the nucleus of the cell indicates that the agent modulates the interaction of the two or more polypeptides.

15. The method of claim 14, wherein the change in location is from a nuclear location to a cytoplasmic location.

16. The method of claim 14, wherein the binding domain of the first polypeptide comprises all or a portion of a binding domain of cyclin dependent kinase 5 (cdk5).

17. The method of claim 16, wherein the binding domain of the second polypeptide comprises all or a portion of a binding domain of p35.

18. The method of claim 16, wherein the binding domain of the second polypeptide comprises all or a portion of a binding domain of p25.

19. The method of claim 14, wherein the binding domain of the first polypeptide comprises all or a portion of a binding domain of from p53.

20. The method of claim 19, wherein the binding domain of the second polypeptide comprises all or a portion of a binding domain of HDM2.

21. A method for identifying the presence of a binding domain in a polypeptide to be assessed, comprising:
a) introducing into a cell a first polypeptide comprising a localization domain, a reporter domain, and a binding domain;
b) introducing into the cell the polypeptide to be assessed, the polypeptide to be assessed comprising a reporter domain, and a localization domain that is different from the localization domain of the first polypeptide;
b) maintaining the cell under conditions in which the first polypeptide interacts with the polypeptide to be assessed when the polypeptide to be assessed comprises a binding domain that is capable of binding to the binding domain of the first polypeptide;
c) determining the cellular location of the polypeptide to be assessed,
wherein if the polypeptide to be assessed co-localizes with the first polypeptide, this indicates that the first polypeptide interacts with the polypeptide to be assessed and that a binding domain is present in the polypeptide to be assessed.

22. The method of claim 21, wherein the polypeptide to be assessed is at least a fragment of an endogenous molecule or at least a fragment of an exogenous molecule.

23. A polypeptide comprising:
a) at least a fragment of a neurodegenerative disease-associated protein, wherein the fragment comprises a binding domain;
b) a reporter domain; and
c) a localization domain.

24. The polypeptide of claim 23, wherein the neurodegenerative disease-associated protein is p25.

25. A composition comprising at least two polypeptides for screening drugs for treatment of a neurodegenerative disease, comprising:
a) a first polypeptide comprising at least a fragment of a neurodegenerative disease-associated protein, wherein the fragment comprises a binding domain, a localization domain, and a reporter domain; and
b) a second polypeptide comprising a binding domain, a localization domain, and a reporter domain,
wherein the localization domain of the second polypeptide is different from the localization domain of the first polypeptide, and wherein the binding domain of the first polypeptide binds to the binding domain of the second polypeptide.

26. The composition of claim 25, wherein the first polypeptide comprises all or a portion of a binding domain of p35 or p25, and the second polypeptide comprises all or a portion of a binding domain of cyclin dependent kinase 5 (cdk5).

27. A polypeptide comprising an amino acid sequence selected from the group consisting of: SEQ ID NOS: 2, 7, 12, 15, 19, 21, 23, 25, 28, 30, 32, 35, and 37.

28. A polypeptide consisting essentially of an amino acid sequence selected from the group consisting of: SEQ ID NOS: 2, 7, 12, 15, 19, 21, 23, 25, 28, 30, 32, 35, and 37.

29. A nucleic acid sequence encoding a sequence selected from the group consisting of: SEQ ID NOS: 2, 7, 12, 15, 19, 21, 23, 25, 28, 30, 32, 35, and 37.

30. A nucleic acid sequence comprising a sequence selected from the group consisting of SEQ ID NOS: 1, 6, 11, 14, 18, 20, 22, 24, 27, 29, 21, 34, and 36.

31. A nucleic acid sequence consisting essentially of a sequence selected from the group consisting of SEQ ID NOS: 1, 6, 11, 14, 18, 20, 22, 24, 27, 29, 21, 34, and 36.

32. A polypeptide comprising a binding domain, a localization domain, and a reporter domain, wherein the binding domain is selected from the group consisting of: SEQ ID NOS: 5, 10, 13, 17, 26, and 38.

33. A polypeptide comprising a binding domain, a localization domain, and a reporter domain, wherein the localization domain is selected from the group consisting of: SEQ ID NOS: 4, 9, 39, 40, 41, 42, 43, 44, and 45.

34. A polypeptide comprising a binding domain, a localization domain, and a reporter domain, wherein the reporter domain is selected from the group consisting of: SEQ ID NOS: 3, 8, 16, and 33.

35. A polypeptide comprising a binding domain, a localization domain, and a reporter domain, wherein
a) the binding domain is selected from the group consisting of: SEQ ID NOS: 5, 10, 13, 17, 26, and 38;
b) the localization domain is selected from the group consisting of: SEQ ID NOS: 4, 9, 39, 40, 41, 42, 43, 44, and 45; and
c) the reporter domain is selected from the group consisting of: SEQ ID NOS: 3, 8, 16, and 33.

36. A vector comprising a nucleic acid sequence encoding a polypeptide, wherein the polypeptide comprises a binding domain, a localization domain, and a reporter domain, wherein
a) the binding domain is selected from the group consisting of: SEQ ID NOS: 5, 10, 13, 17, 26, and 38;
b) the localization domain is selected from the group consisting of: SEQ ID NOS: 4, 9, 39, 40, 41, 42, 43, 44, and 45; and
c) the reporter domain is selected from the group consisting of: SEQ ID NOS: 3, 8, 16, and 33.

37. A host cell comprising a vector, wherein the vector comprises a nucleic acid sequence encoding a polypeptide, wherein the polypeptide comprises a binding domain, a localization domain, and a reporter domain, wherein
a) the binding domain is selected from the group consisting of: SEQ ID NOS: 5, 10, 13, 17, 26, and 38;
b) the localization domain is selected from the group consisting of: SEQ ID NOS: 4, 9, 39, 40, 41, 42, 43, 44, and 45; and
c) the reporter domain is selected from the group consisting of: SEQ ID NOS: 3, 8, 16, and 33.

38. A kit comprising:
a) a nucleic acid which encodes a polypeptide comprising a binding domain, a localization domain, and a reporter domain, wherein
i) the binding domain is selected from the group consisting of: SEQ ID NOS: 5, 10, 13, 17, 26, and 38;
ii) the localization domain is selected from the group consisting of: SEQ ID NOS: 4, 9, 39, 40, 41, 42, 43, 44, and 45; and
ii) the reporter domain is selected from the group consisting of: SEQ ID NOS: 3, 8, 16, and 33;
b) a vector comprising a nucleic acid sequence encoding a polypeptide, wherein the polypeptide comprises a binding domain, a localization domain, and a reporter domain, wherein
i) the binding domain is selected from the group consisting of: SEQ ID NOS: 5, 10, 13, 17, 26, and 38;
ii) the localization domain is selected from the group consisting of: SEQ ID NOS: 4, 9, 39, 40, 41, 42, 43, 44, and 45; and
iii) the reporter domain is selected from the group consisting of: SEQ ID NOS: 3, 8, 16, and 33;
c) a host cell comprising a vector, wherein the vector comprises a nucleic acid sequence encoding a polypeptide, wherein the polypeptide comprises a binding domain, a localization domain, and a reporter domain, wherein
i) the binding domain is selected from the group consisting of: SEQ ID NOS: 5, 10, 13, 17, 26, and 38;
ii) the localization domain is selected from the group consisting of: SEQ ID NOS: 4, 9, 39, 40, 41, 42, 43, 44, and 45; and
iii) the reporter domain is selected from the group consisting of: SEQ ID NOS: 3, 8, 16, and 33;
d) or a combination thereof;
and instructions for use.
